(19) United States
(12) Patent Application Publication
(10) Pub. No.: US 2008/0241129 A1
(43) Pub. Date: Oct. 2, 2008

(54) ADIPOCYTE SECRETED PROTEIN CCDC80 IS A POTENT STIMULATOR OF BONE FORMATION

(75) Inventors: Ruth E. Gimeno, Wellesley, MA (US); Frederic Tremblay, Cambridge, MA (US); Shoichi Fukayama, Brookline, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/031,265

(22) Filed: Feb. 14, 2008

Related U.S. Application Data
(60) Provisional application No. 60/901,888, filed on Feb. 16, 2007.

(51) Int. Cl. A61K 31/7088; A61K 38/16; A61K 31/4439; A61P 19/00; G01N 33/53; A61K 39/395

(52) U.S. Cl. 424/130.1; 514/12; 514/44; 514/342; 435/7.1

(57) ABSTRACT

Disclosed herein are methods of using Ccdc80, Ccdc80 mimics, or Ccdc80 agonists or antagonists in the treatment of bone disorders.

### CROSS-REFERENCE TO RELATED APPLICATIONS

This application claims the benefit of U.S. Provisional Application No. 60/901,888, filed Feb. 16, 2007, the entire contents of which are incorporated herein in their entirety.

### FIELD OF THE INVENTION

The present invention relates to use of Ccdc80 in relation to bone formation and remodeling.

### BACKGROUND OF THE INVENTION

Ccdc80 (also termed mouse URB (up-regulated in bombesin receptor subtype-3 knockout mice), human DRO1 (down-regulated by oncogenes 1), rat SSG1 (steroid-sensitive gene 1), chicken EQUARIN) was initially described as a ubiquitously expressed gene that is up-regulated in the brown adipose tissue of bombesin receptor subtype-3 knock-out mice (Aoki K et al., Biochem. Biophys. Res. Commun. 290:1282-88 (2002)). Subsequently, human Ccdc80 was shown to be expressed in bone marrow stromal cells and to be down-regulated during differentiation of these cells into osteoblasts (Liu Y. et al., Biochem. Biophys. Res. Commun. 322:497-507 (2004)). Ccdc80 mRNA and protein were also shown to be present in chondrocytes and associated extracellular matrix during mouse embryo development (Liu Y. et al., Biochem. Biophys. Res. Commun. 322:497-507 (2004)). A chicken ortholog of Ccdc80 was found to be expressed exclusively in the lens equatorial region (Mu H et al., Mech. Dev. 120:143-55 (2003)). While mouse Ccdc80 and chicken Ccdc80 have been demonstrated to be secreted proteins (Liu Y. et al., Biochem. Biophys. Res. Commun. 322:497-507 (2004); Mu H et al., Mech. Dev. 120:143-55 (2003)), human Ccdc80 was reported to be localized intracellularly with no appreciable amounts being secreted (Bommer G T et al., J. Biol. Chem. 280:7962-75 (2005)). Human Ccdc80 was found to be down-regulated in cells neoplastically transformed with β-catenin, and overexpression of Ccdc80 in these cells was able to inhibit growth, leading to the designation of Ccdc80 as a candidate tumor suppressor gene (Bommer G T et al., J. Biol. Chem. 280:7962-75 (2005)). To date, no other functional data have been reported for any mammalian Ccdc80orthologs.

### SUMMARY OF THE INVENTION

One aspect is for a method for the treatment of a mammal suffering from a bone disorder comprising administering to the mammal in need thereof a therapeutically effective amount of an agent that modulates the expression or activity of the Ccdc80 gene or Ccdc80 protein. In some embodiments, the mammal is a human. In some embodiments, the bone disorder being treated is osteoporosis, rickets, osteomalacia, chronic renal failure, hyperparathyroidism, osteopenia, Paget's disease, or a bone lesion.

An agent that modulates the expression or activity of the Ccdc80 gene or Ccdc80 protein may be alternatively referred to herein as a “Ccdc80 modulator”. The agent may be a compound, a protein, a polypeptide, an antibody, an aptamer, or a polynucleotide. In some embodiments, the agent is a human, mouse, rat, or chicken Ccdc80 polypeptide. In some other embodiments, the agent is a Ccdc80 peptidomimetic or a Ccdc80 agonist. In still further embodiments, the agent is a human, mouse, rat, or chicken Ccdc80 polynucleotide. In certain embodiments, the agent is a compound. In some embodiments, the compound is rosiglitazone.

In some embodiments, the agent directly modulates the expression or activity of the Ccdc80 gene or Ccdc80 protein. This aspect will be described in further detail below. The present invention further provides a method of identifying a Ccdc80 receptor comprising: a) providing Ccdc80 polypeptide to a cell suspected of containing a Ccdc80 receptor; b) identifying specific binding of the Ccdc80 polypeptide to the cell; and c) isolating the source of the specific binding. The method may be performed in vivo or in vitro. In some embodiments, the cell employed in this method is an osteoblast, osteoclast, or hepatocyte. The Ccdc80 polypeptide may be provided to the cell in step a) by contacting the Ccdc80 polypeptide with the cell. In other embodiments, the Ccdc80 polypeptide may be provided to the cell in step a) by expression of the Ccdc80 polypeptide in the cell. The Ccdc80 polypeptide may be expressed from an expression vector comprising a Ccdc80 polynucleotide. In some embodiments, the expressed Ccdc80 polypeptide is secreted by the cell suspected of containing a Ccdc80 receptor. In alternative embodiments, the Ccdc80 polypeptide provided in step a) of this method is provided by expression and secretion of the Ccdc80 polypeptide by a different cell.

An additional aspect relates to a method of activating osteoblasts and/or enhancing bone remodeling comprising contacting an osteoblast with an effective amount of an agent that modulates the expression or activity of the Ccdc80 gene or Ccdc80 protein.

Another aspect is for a method of screening for Ccdc80 mimics comprising: a) providing a candidate mimic and a Ccdc80 polypeptide; and b) determining whether the candidate mimic competes with Ccdc80 polypeptide in an assay designed to assess Ccdc80 polypeptide activity; wherein the Ccdc80 polypeptide activity is activation of osteoblasts and/or enhancement of bone remodeling. In some embodiments, the assay is performed in a calvaria organ culture. In some other embodiments, the assay is a measurement of total bone area. In some further embodiments, the assay is a measurement of osteoblast morphology.

A further aspect relates to a method of screening for modulators that affect Ccdc80 activity comprising: a) providing a candidate modulator and a Ccdc80 polypeptide; and b) determining whether the candidate modulator interferes with or enhances Ccdc80 activity; wherein the Ccdc80 activity is activation of osteoblasts and/or enhancement of bone remodeling.

Another aspect of the present invention relates to the use of an agent that modulates the expression or activity of the Ccdc80 gene or Ccdc80 protein in the manufacture of a medicament for the treatment of a bone disorder. In some embodiments, such a medicament is for the treatment of a bone disorder selected from the following: osteoporosis, rickets, osteomalacia, chronic renal failure, hyperparathyroidism, osteopenia, Paget's disease, or a bone lesion.

Other objects and advantages of the present invention will become apparent to those skilled in the art upon reference to the detailed description that hereinafter follows.

### BRIEF DESCRIPTION OF THE FIGURES

FIG. 1A is a Western blot using anti-Flag antibody showing that Flag-reactive material is present in both the cell lysate and the supernatant. Lane 1: empty vector (lysate); lane 2: human Ccdc80-FLAG (lysate); lane 3: empty vector (supernatant); lane 4: human Ccdc80-FLAG (supernatant).

FIG. 1B is a commassie stained gel of the supernatant from Ccdc80- and control-transfected cells. (See Example 1).

FIG. 2A is a bar graph showing the effects of Ccdc80 on total bone area and number of osteoblasts. Calvaria were prepared from 4-day old neonatal pups. Calvaria then were incubated for 7 days without (control), 5% or 1% control conditioned medium (CM) samples, or with 5% or 1% human Ccdc80 CM samples. Medium was changed on day 4. After organ culture, calvaria were fixed in 10% neutral phosphate buffered formaldehyde, embedded in paraffin block, sectioned at 4 mm, and stained with hematoxylin and eosin for histomorphometric analysis. Each bar gives the Mean ±SE for 5 cultures. Control values were 0.0048930±0.0005159 mm² (Total Bone Area) and 117±9.0 (Number of Osteoblasts). *, P<0.05; **, P<0.01; ***, P<0.001; ***, P<0.0001. FIG. 2B shows representative images of calvaria from FIG. 2A. Left, control calvaria; middle, calvaria incubated with 1% control CM; right, calvaria incubated with 1% Ccdc80 CM. (See Example 2).

FIG. 3A is a bar graph showing the effects of FLAG-purified Ccdc80 protein on total bone area and number of osteoblasts. Calvaria were prepared from 4-day old neonatal pups. Calvaria then were incubated 7 days without (Control), or 1% FLAG-purified Ccdc80 protein, or with 1% FLAG purification buffer. Medium was changed on day 4. After organ culture, calvaria were fixed in 10% neutral phosphate buffered formaldehyde, embedded in paraffin block, section at 4 mm, and stained with hematoxylin and eosin for histomorphometric analysis. Each bar gives the Mean ±SE for 4 (FLAG purification buffer) or 5 (other groups) cultures. Control values were 0.0042996±0.0003713 mm² (total bone area) and 118±10.0 (Number of Osteoblasts). ***, P<0.001. FIG. 3B shows representative images of calvaria from FIG. 3A. Left, control calvaria; middle, calvaria incubated with 1% FLAG-purification buffer; right, calvaria incubated with 1% FLAG-purified Ccdc80 protein. (See Example 3).

FIG. 4 is a bar graph showing effects of Ccdc80 on total bone area and number of osteoblasts. Each bar gives the Mean ±SE for 4 or 5 cultures. Control values were 0.0048633±0.0003492 mm² (Total Bone Area) and 75±5.9 (Number of Osteoblasts). (a), Osteoblasts were activated. *, P<0.05; **, P<0.01; ***, P<0.001. (See Example 3).

FIG. 5 is a bar graph showing effects of Ccdc80 gene transfer on total bone area and number of osteoblasts. Calvaria were prepared from 4-day old neonatal pups. Calvaria then were infected without (Control) or with various particle numbers of Ccdc80-adenovirus as shown in the Figure. Fresh treatments were added when medium was changed on day 4. After organ culture, calvaria were fixed in 10% neutral phosphate buffered formaldehyde, embedded in paraffin block, sectioned at 4 mm, and stained with hematoxylin and Eosin for histomorphoemetric analysis. Each bar gives the Mean ±SE for 6 (Control) or 5 (other groups) cultures. Control values were 0.0045174±0.00002772 mm² (Total Bone Area) and 77±2.1 (Number of Osteoblasts). (a), Osteoblasts were activated. (b), Active bone remodeling. *, P<0.05; ***, P<0.001; ****, P<0.0001 vs. Control. (See Example 3).

FIG. 6 is a bar graph showing the effect of Ccdc80 on BMP-mediated transcriptional activity in C2C12 cells. (See Example 4).

FIG. 7 is a bar graph showing the effect of Ccdc80 on β-catenin-mediated transcriptional activity in C2C12 cells. (See Example 5).

FIG. 8 is a bar graph showing the effect of Ccdc80 on β-catenin-mediated transcriptional activity in HepG2 cells. (See Example 5).

FIG. 9 shows the effect of Ccdc80 on β-catenin protein expression in HepG2 cells. (See Example 5).

FIG. 10 shows the effect of Ccdc80 on Akt phosphorylation and protein expression in HepG2 cells. (See Example 6).

FIG. 11 shows the effect of Ccdc80 on p38 MAPK, ERK-1/2 and JNK-1/2 phosphorylation in HepG2 cells. (See Example 6).

### BRIEF DESCRIPTION OF SEQUENCES

SEQ ID NO:1 is a forward Ccdc80 primer.

SEQ ID NO:2 is a reverse Ccdc80 primer.

### DETAILED DESCRIPTION OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); U.S. Pat. No. 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshney, Alan R. Liss, Inc., 1987); Immobilized Cells and Enzymes (IRL Press, 1986); B. Perbal, A Practical Guide to Molecular Cloning (1984); Methods in Enzymology (Academic Press, Inc., N.Y.); Gene Transfer Vectors for Mammalian Cells (J. H. Miller and M. P. Calos eds., 1987, Cold Spring Harbor Laboratory); Methods in Enzymology, Vols. 154 and 155 (Wu et al. eds.), Immunochemical Methods in Cell and Molecular Biology (Mayer and Walker, eds., Academic Press, London, 1987); Handbook of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Here, Applicants are the first to describe a functional role for Ccdc80 in bone formation. Applicants' data suggest that Ccdc80 protein or a derivative of Ccdc80 protein may be useful as a therapeutic agent in bone disorders where bone formation lags behind bone resorption, such as osteoporosis. Applicants' data also provide an assay that can be used to identify a receptor for Ccdc80 as well as protein fragments, peptides, antibodies, or small molecules that mimic the function of Ccdc80.

Applicants also report that Ccdc80 is a potent stimulator of bone formation. Using a neonatal calvaria organ culture assay, Applicants show that incubation with Ccdc80-containing supernatant or purified Ccdc80 protein increases total bone area by 50-100%. While the number of osteoblasts was unaltered, Ccdc80-treated calvaria were characterized by activated osteoblasts and by features typical of enhanced bone remodeling (both bone formation and bone resorption). Induction of active bone remodeling is the requirement for bone formation stimulated by parathyroid hormone (PTH), the only clinically approved osteogenic agent for osteoporosis treatment (Neer R M et al., N. Engl. J. Med. 344:1434-41 (2001); Finkelstein J S et al., N. Engl. J. Med. 349:1216-26 (2003); Cosman F et al., N. Engl. J. Med. 353:566-75 (2005)). The phenotype observed upon addition of Ccdc80 to calvarial cultures was also similar to what has been observed with treatment of these cultures with bone morphogenetic protein-2 (BMP-2), a well-known bone forming agent (Traianedes K et al., Endocrinology 139:3178-84 (1998)).

### I. DEFINITIONS

In the context of this disclosure, a number of terms shall be utilized.

As used herein, the term “about” or “approximately” means within 20%, preferably within 10%, and more preferably within 5% of a given value or range.

“Bone modulation” or “modulation of bone formation” refers to the ability to affect any of the physiological processes involved in bone remodeling, as will be appreciated by one skilled in the art, including, for example, bone resorption and appositional bone growth, by, inter alia, osteoclastic and osteoblastic activity, and may comprise some or all of bone formation and development as used herein. Bone is a dynamic tissue that is continually adapting and renewing itself through the removal of old or unnecessary bone by osteoclasts and the rebuilding of new bone by osteoblasts. The nature of the coupling between these processes is responsible both for the modeling of bone during growth as well as the maintenance of adult skeletal integrity through remodeling and repair to meet the everyday needs of mechanical usage. There are a number of diseases of bone that result from an uncoupling of the balance between bone resorption and formation. With aging there is a gradual “physiologic” imbalance in bone turnover, which is particularly exacerbated in women due to menopausal loss of estrogen support, that leads to progressive loss of bone. The reduction in bone mass and deterioration in bone architecture results in an increase in bone fragility and susceptibility to spontaneous fractures. For every 10 percent of bone that is lost, the risk of fracture doubles.

“Bone remodeling” as used herein refers to the tightly coupled continuous biological process in living bones, characterized by ‘bone resorption’ by osteoclasts and ‘bone formation’ by osteoblasts; that is, the process that allows the replacement of old bone with new bone.

An “antibody” includes an immunoglobulin molecule capable of binding an epitope present on an antigen. As used herein, the term encompasses not only intact immunoglobulin molecules such as monoclonal and polyclonal antibodies, but also anti-idotypic antibodies, mutants, fragments, fusion proteins, bi-specific antibodies, humanized proteins, and modifications of the immunoglobulin molecule that comprise an antigen recognition site of the required specificity.

The term “Ccdc80” or “coiled-coil domain containing 80” is used herein interchangeably with its other common names URB, DRO1, SSG1, and EQUARIN. Exemplary GenBank® accession numbers for Ccdc80 sequences include the following: human (Homo sapiens, NM_{—}199511), mouse (Mus musculus, NM_{—}026439), rat (Rattus norvegicus, NM_{—}022543), chicken (Gallus gallus, NM_{—}204431).

The term “cDNA” includes complementary DNA, that is mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into vectors. The library can then be probed for the specific cDNA (and thus mRNA) of interest.

As used herein, a Ccdc80 “chimeric protein” or “fusion protein” comprises a Ccdc80 polypeptide operably linked to a non-Ccdc80 polypeptide. A “Ccdc80 polypeptide” refers to a polypeptide having an amino acid sequence corresponding to Ccdc80 polypeptide, whereas a “non-Ccdc80 polypeptide” refers to a polypeptide having an amino acid sequence corresponding to a protein which is not substantially homologous to the Ccdc80 protein, for example, a protein which is different from the Ccdc80 protein and which is derived from the same or a different organism. Within a Ccdc80 fusion protein, the Ccdc80 polypeptide can correspond to all or a portion of a Ccdc80 protein. In a preferred embodiment, a Ccdc80 fusion protein comprises at least one biologically active portion of a Ccdc80 protein. Within the fusion protein, the term “operably linked” is intended to indicate that the Ccdc80 polypeptide and the non-Ccdc80 polypeptide are fused in-frame to each other. The non-Ccdc80 polypeptide can be fused to the N-terminus or C-terminus of the Ccdc80 polypeptide.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a Ccdc80 polypeptide is preferably replaced with another amino acid residue from the same side chain family.

The terms “effective amount”, “therapeutically effective amount”, and “effective dosage” as used herein, refer to the amount of a molecule that, when administered to a mammal in need, is effective to at least partially ameliorate conditions related to, for example, a bone disorder (for example, osteoporosis or osteopenia), or is effective to at least partially enhance, for example, bone remodeling and/or osteoblast activity. Moreover, such treatment will be indicated in the treatment of bone lesions, particularly bone fractures, for bone remodeling in the healing of such lesions. For example, persons predisposed to or suffering from stress fractures (i.e., the accumulation of stress-induced microfractures, eventually resulting in a true fracture through the bone cortex) may be identified and/or treated by means of the invention. Moreover, the methods and compositions of the invention will be of use in the treatment of secondary osteoporosis, where the course of therapy involves bone remodeling, such as endocrine conditions accompanying corticosteroid administration, hyperthyroidism, hypogonadism, hematologic malignancies, malabsorption and alcoholism, as well as disorders associated with vitamin D and/or phosphate metabolism, such as osteomalacia and rickets, and diseases characterized by abnormal or disordered bone remodeling, such as Paget's disease, and in neoplasms of bone, which may be benign or malignant.

Individuals with bone mineral density (BMD) in the spine or proximal femur 2.5 or more standard deviations below normal peak bone mass are classified as osteoporotic. However, osteopenic individuals with BMD between 1 and 2.5 standard deviations below the norm are clearly at risk of suffering bone loss related disorders. Bone modulation may be assessed by measuring parameters such as BMD and bone mineral content (BMC) by pDXA X-ray methods, bone size, thickness or volume as measured by X-ray, bone formation rates as measured for example by calcien labeling, total, trabecular, and mid-shaft density as measured by pQCT and/or CT methods, connectivity and other histological parameters as measured by ACT methods, mechanical bending and compressive strengths as preferably measured in femur and vertebrae respectively. Due to the nature of these measurements, each may be more or less appropriate for a given situation as the skilled practitioner will appreciate. Furthermore, parameters and methodologies such as a clinical history of freedom from fracture, bone shape, bone morphology, connectivity, normal histology, fracture repair rates, and other bone quality parameters are known and used in the art. Most preferably, bone quality may be assessed by the compressive strength of vertebra when such a measurement is appropriate. Bone modulation may also be assessed by rates of change in the various parameters. Most preferably, bone modulation is assessed at more than one age. “Normal bone density” refers to a bone density within two standard deviations of a Z score of O in the context of the HBM linkage study. In a general context, the range of normal bone density parameters is determined by routine statistical methods. A normal parameter is within about 1 or 2 standard deviations of the age and sex normalized parameter, preferably about 2 standard deviations. A statistical measure of meaningfulness is the P value which can represent the likelihood that the associated measurement is significantly different from the mean. Significant P values are P 0.05, 0.01, 0.005, and 0.001, preferably at least P 0.01.

The term “modulate” encompasses either a decrease or an increase in activity depending on the target molecule. For example, a Ccdc80 modulator is considered to modulate the activity of Ccdc80 if the presence of the Ccdc80 modulator results in an increase or decrease in Ccdc80 activity. The term “modulates” as in “an agent that modulates the expression or activity of the Ccdc80 gene or Ccdc80 protein” means that the agent directly or indirectly modulates the expression or activity.

As used herein, the term “directly modulates” as in “an agent that directly modulates the expression or activity of the Ccdc80 gene or Ccdc80 protein” means that the agent or a derivative thereof directly binds or directly interacts with a Ccdc80 protein, a Ccdc80 receptor or a Ccdc80 polynucleotide (e.g., gene or mRNA encoded by a gene), thereby modulating the functional activity of Ccdc80 protein. For example, and without being bound to any one theory, the functional activity of Ccdc80 protein may be stimulated by an agent that directly interacts with Ccdc80 protein, a Ccdc80 receptor or a Ccdc80 polynucleotide. Such stimulatory agents may include, but are not limited to, Ccdc80 polypeptides, Ccdc80 peptidomimetics, Ccdc80 agonists, or small molecules. Agents found to stimulate Ccdc80 activity would be useful to treat bone disorders characterized by insufficient production of Ccdc80 protein or production of Ccdc80 protein forms which have decreased or aberrant activity relative to Ccdc80 wild-type protein, for example. Alternatively, the functional activity of Ccdc80 protein may be sequestered or inhibited by an agent that directly interacts with Ccdc80 protein, such as a neutralizing Ccdc80 antibody, or a small molecule. As another example, translation of Ccdc80 mRNA may be prevented or reduced by an agent, such as a Ccdc80-specific RNAi, e.g., a small interfering RNA (siRNA) or a short hairpin RNA (shRNA), that specifically silences the expression of the Ccdc80 gene. Agents found to inhibit Ccdc80 activity would be useful to treat bone disorders characterized by excessive production of Ccdc80 protein, for example. In some embodiments, the agent “directly modulates” by binding to the Ccdc80 protein, Ccdc80 RNA or promoter of the Ccdc80 gene. In some embodiments, the Ccdc80 modulator is not rosiglitazone. In some further embodiments, the Ccdc80 modulator is not a glitazone, such as, but not limited to, the thiazolidinedione agents.

For example, in copending, commonly-owned U.S. application Ser. No. ______, filed Feb. 13, 2008, entitled “The Secreted Protein Ccdc80 Regulates Adipocyte Differentiation”, rosiglitazone was shown to modulate Ccdc80. However, since rosiglitazone is an anti-diabetic drug in the thiazolidinedione class of drugs and, like other thiazolidinediones, binds the intracellular receptor class of the peroxisome proliferator-activated receptors (PPARs), specifically PPARγ (i.e., rosiglitazone is a selective ligand of PPARγ and has no PPARα-binding action), it does not directly modulate Ccdc80.

As used herein, the term “expression” includes the process by which polynucleotides are transcribed into mRNA. As used herein, the term “expression” also includes the process by which an mRNA is translated into an amino acid sequence. As used herein, the term “expression” further includes the process by which polynucleotides are transcribed into mRNA and translated into peptides, polypeptides, or proteins. As used herein, the phrase “modulates the expression or activity of the Ccdc80 gene or Ccdc80 protein” is intended to include an increase or decrease in mRNA or polypeptide levels, as well as an increase or decrease in protein activity. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T., Molecular Cloning: A Laboratory Manual, 2nd ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

The term “expression construct” means any double-stranded DNA or double-stranded RNA designed to transcribe an RNA, e.g., a construct that contains at lease one promoter operably linked to a downstream gene or coding region of interest (e.g., a cDNA or genomic DNA fragment that encodes a protein, or any RNA of interest). Transfection or transformation of the expression construct into a recipient cell allows the cell to express RNA or protein encoded by the expression construct. An expression construct may be a genetically engineered plasmid, virus, or an artificial chromosome derived from, for example, a bacteriophage, adenovirus, retrovirus, poxvirus, or herpesvirus. An expression construct can be replicated in a living cell, or it can be made synthetically. For purposes of this application, the terms “expression construct”, “expression vector”, “vector”, and “plasmid” are used interchangeably to demonstrate the application of the invention in a general, illustrative sense, and are not intended to limit the invention to a particular type of expression construct. Further, the term expression construct or vector is intended to also include instances wherein the cell utilized for the assay already endogenously comprises such DNA sequence.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

The term “genetically modified” includes a cell containing and/or expressing a foreign gene or nucleic acid sequence which in turn modifies the genotype or phenotype of the cell or its progeny. This term includes any addition, deletion, or disruption to a cell's endogenous nucleotides.

The term “gene product” as used herein, unless otherwise indicated, refers to a product produced by a gene when that gene is transcribed or translated. A “gene product” may be any transcription or translational product derived from a specific gene locus. Typically, the term refers to a nucleic acid, such as, for example, a messenger RNA, or a protein, or a polypeptide. A “gene product” includes an amino acid (e.g., peptide or polypeptide) generated when a gene is transcribed and translated.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is such an element operably associated with a different gene than the one it is operably associated with in nature.

The term “homologous” as used herein refers to the sequence similarity between two polymeric molecules, e.g., between two nucleic acid molecules, e.g., two DNA molecules or two RNA molecules, or between two polypeptide molecules. When a nucleotide or amino acid position in both of the two molecules is occupied by the same monomeric nucleotide or amino acid, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′ATTGCC3′ and 5′TATGCG3′ share 50% homology. By the term “substantially homologous” as used herein, is meant DNA or RNA which is about 50% homologous, in another embodiment about 60% homologous, in another embodiment about 70% homologous, in another embodiment about 80% homologous, in another embodiment about 85% homologous, in another embodiment about 90% homologous, in another embodiment about 95% homologous, to the desired nucleic acid.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100). The percent identity between the two sequences is a function of the number of identical positions shared by the sequences, taking into account the number of gaps, and the length of each gap, which are introduced for optimal alignment of the two sequences.

The comparison of sequences and determination of percent identity between two sequences can be accomplished using a mathematical algorithm. A non-limiting example of a mathematical algorithm utilized for comparison of sequences is the algorithm of Karlin S and Altschul S F, Proc. Natl. Acad. Sci. USA 87:2264-68 (1990), modified as in Karlin S and Altschul S F, Proc. Natl. Acad. Sci. USA 90:5873-77 (1993). Such an algorithm is incorporated into the NBLAST and XBLAST programs (version 2.0) of Altschul S F et al., J. Mol. Biol. 215:403-10 (1990). BLAST nucleotide searches can be performed with the NBLAST program score=100, wordlength=12 to obtain homologous nucleotide sequences. BLAST protein searches can be performed with the XBLAST program, score=50, wordlength=3 to obtain amino acid sequences homologous to the protein molecules of the invention. To obtain gapped alignments for comparison purposes, Gapped BLAST can be utilized as described in Altschul S F et al., Nucleic Acids Res. 25:3389-3402 (1997). When utilizing BLAST and Gapped BLAST programs, the default parameters of the respective programs (e.g., XBLAST and NBLAST) can be used. Another preferred, non-limiting algorithm utilized for the comparison of sequences is the algorithm of Myers E W and Miller W, Comput. Appl. Biosci. 4:11-17 (1988). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Another non-limiting example of a mathematical algorithm utilized for the alignment of protein sequences is the Lipman-Pearson algorithm (Lipman D J and Pearson W R, Science 227:1435-41 (1985)). When using the Lipman-Pearson algorithm, a PAM250 weight residue table, a gap length penalty of 12, a gap penalty of 4, and a Kutple of 2 can be used. A preferred, non-limiting example of a mathematical algorithm utilized for the alignment of nucleic acid sequences is the Wilbur-Lipman algorithm (Wilbur W J and Lipman D J, Proc. Natl. Acad. Sci. USA 80:726-30 (1983)). When using the Wilbur-Lipman algorithm, a window of 20, gap penalty of 3, Ktuple of 3 can be used. Both the Lipman-Pearson algorithm and the Wilbur-Lipman algorithm are incorporated, for example, into the MEGALIGN program (e.g., version 3.1.7) which is part of the DNASTAR sequence analysis software package.

Additional algorithms for sequence analysis are known in the art, and include ADVANCE and ADAM, described in Torelli A and Robotti C A, Comput. Appl. Biosci. 10:3-5 (1994); and FASTA, described in Pearson W R and Lipman D J, Proc. Natl. Acad. Sci. USA 85:2444-48 (1988).

In one embodiment, the percent identity between two amino acid sequences is determined using the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package, using a NWSgapdna. CMP matrix and a gap weight of 40, 50, 60, 70, or 80 and a length weight of 1, 2, 3, 4, 5, or 6.

Protein alignments can also be made using the Geneworks global protein alignment program (e.g., version 2.5.1) with the cost to open gap set at 5, the cost to lengthen gap set at 5, the minimum diagonal length set at 4, the maximum diagonal offset set at 130, the consensus cutoff set at 50% and utilizing the Pam 250 matrix.

A “host cell” is intended to include any individual cell or cell culture which can be or has been a recipient for vectors or for the incorporation of exogenous nucleic acid molecules, polynucleotides, and/or proteins. It also is intended to include progeny of a single cell. The progeny may not necessarily be completely identical (in morphology or in genomic or total DNA complement) to the original parent cell due to natural, accidental, or deliberate mutation. The cells may be prokaryotic or eukaryotic, and include but are not limited to bacterial cells, yeast cells, insect cells, animal cells, and mammalian cells, e.g., murine, rat, simian, or human cells.

“Hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. The stringency of a hybridization reaction includes the difficulty with which any two nucleic acid molecules will hybridize to one another. Under stringent conditions, nucleic acid molecules at least 65%, 70%, 75%, 80%, 85%, 90%, 95% or more identical to each other remain hybridized to each other, whereas molecules with low percent identity cannot remain hybridized. A preferred, non-limiting example of highly stringent hybridization conditions are hybridization in 6× sodium chloride/sodium citrate (SSC) at about 45° C., followed by one or more washes in 0.2×SSC, 0.1% SDS at 50° C., preferably at 55° C., more preferably at 60° C., and even more preferably at 65° C.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide if hybridization can occur between one of the strands of the first polynucleotide and the second. “Complementarity” or homology is quantifiable in terms of the proportion of bases in opposing strands that are expected to hydrogen bond with each other, according to generally accepted base-pairing rules.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes, for example, a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “mammal” refers to a human, a non-human primate, canine, feline, bovine, ovine, porcine, murine, or other veterinary or laboratory mammal. Those skilled in the art recognize that a therapy which reduces the severity of a pathology in one species of mammal is predictive of the effect of the therapy on another species of mammal.

As used herein, a “naturally-occurring” nucleic acid molecule refers to an RNA or DNA molecule having a nucleotide sequence that occurs in nature (e.g., encodes a natural protein).

The term “operably linked” means that a nucleic acid molecule, e.g., DNA, and one or more regulatory sequences (e.g., a promoter or portion thereof) are connected in such a way as to permit transcription of mRNA from the nucleic acid molecule or permit expression of the product (i.e., a polypeptide) of the nucleic acid molecule when the appropriate molecules are bound to the regulatory sequences. Within a fusion construct, the term “operably linked” is intended to indicate that the Ccdc80 polynucleotide and a non-Ccdc80 polynucleotide are fused in-frame to each other. The non-Ccdc80 polynucleotide can be fused 3′ or 5′ to the Ccdc80 polynucleotide.

As used herein, the terms “polynucleotide” and “oligonucleotide” are used interchangeably, and include polymeric forms of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. In some embodiments, a polynucleotide encoding Ccdc80 may be used to treat bone disorders characterized by insufficient production of Ccdc80 protein, or by production of Ccdc80 protein forms which have decreased activity compared to Ccdc80 wild type protein. In some other embodiments, a small interfering RNA (siRNA) or a short hairpin RNA (shRNA), that specifically silences the expression of the Ccdc80 gene, may be used to treat bone disorders characterized by excessive production of Ccdc80 protein, for example. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule.

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set of primers” consisting of an “upstream” and a “downstream” primer, and a catalyst of polymerization, such as a DNA polymerase, and typically a thermally-stable polymerase enzyme. Methods for PCR are well known in the art, and are taught, for example, in MacPherson M et al., PCR: A Practical Approach, IRL Press at Oxford University Press (1991). All processes of producing replicate copies of a polynucleotide, such as PCR or gene cloning, are collectively referred to herein as “replication”. A primer can also be used as a probe in hybridization reactions, such as Southern or Northern blot analyses (see, e.g., Sambrook, J., Fritsh, E. F., and Maniatis, T. Molecular Cloning: A Laboratory Manual, 2nd ed., Cold Spring Harbor Laboratory, Cold Spring Harbor Laboratory Press, Cold Spring Harbor, N.Y., 1989).

A “probe” when used in the context of polynucleotide manipulation includes an oligonucleotide that is provided as a reagent to detect a target present in a sample of interest by hybridizing with the target. Usually, a probe will comprise a label or a means by which a label can be attached, either before or subsequent to the hybridization reaction. Suitable labels include, but are not limited to, radioisotopes, fluorochromes, chemiluminescent compounds, dyes, and proteins, including enzymes.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined, for example, by mapping with nuclease S1), as well as protein binding domains (consensus sequences) responsible for the binding of RNA polymerase. In one embodiment, the promoter region can be selected from the group consisting of CMV, RSV, SV40, EF-1a, CMV-β-Actin, histone, type I collagen, TGFβ1, SX2, cfos/cjun, Cbfal, Fra/Jun, D1×5, osteocalcin, osteopontin, bone sialoprotein, collagenase, TRAP BC, and TRAP C promoter regions. Bone-specific promoters often preferred for recombinant vector delivery approaches.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure; and more preferably still at least 99% pure. Purity can be evaluated by chromatography, gel electrophoresis, immunoassay, composition analysis, biological assay, and other methods known in the art.

The term “test compound” includes compounds with known chemical structure but not necessarily with a known function or biological activity. Test compounds could also have unidentified structures or be mixtures of unknown compounds, for example from crude biological samples such as plant extracts. Large numbers of compounds could be randomly screened from “chemical libraries” which refers to collections of purified chemical compounds or collections of crude extracts from various sources. The chemical libraries may contain compounds that were chemically synthesized or purified from natural products. The compounds may comprise inorganic or organic small molecules or larger organic compounds such as, for example, proteins, peptides, glycoproteins, steroids, lipids, phospholipids, nucleic acids, and lipoproteins. The amount of compound tested can very depending on the chemical library, but, for purified (homogeneous) compound libraries, 10 μM is typically the highest initial dose tested. Methods of introducing test compounds to cells are well known in the art.

### II. ISOLATED POLYNUCLEOTIDES ENCODING CCDC80 OR PORTIONS THEREOF

In practicing the methods of the invention, various agents can be used to modulate the activity and/or expression of Ccdc80 in a cell. In one embodiment, an agent is a nucleic acid molecule encoding a Ccdc80 polypeptide or a portion thereof. Exemplary GenBank® accession numbers for Ccdc80 sequences include the following: human (Homo sapiens, NM_{—}199511), mouse (Mus musculus, NM_{—}026439), rat (Rattus norvegicus, NM_{—}022543), chicken (Gallus gallus, NM_{—}204431).

A polynucleotide can be amplified using cDNA, mRNA or alternatively, genomic DNA, as a template and appropriate oligonucleotide primers according to standard PCR amplification techniques. The polynucleotide so amplified can be cloned into an appropriate vector and characterized by DNA sequence analysis. Furthermore, oligonucleotides corresponding to Ccdc80 polynucleotide sequences can be prepared by standard synthetic techniques, e.g., using an automated DNA synthesizer.

Moreover, a Ccdc80 polynucleotide can comprise only a portion of a Ccdc80 full-length polynucleotide sequence, for example, a fragment which can be used as a probe or primer or a fragment encoding a biologically active portion of a Ccdc80 protein. The polynucleotide sequence determined from the cloning of Ccdc80 genes allows for the generation of probes and primers designed for use in identifying and/or cloning other Ccdc80 family members, as well as Ccdc80 family homologues from other species.

The probe/primer typically comprises a substantially purified oligonucleotide. In one embodiment, the oligonucleotide comprises a region of nucleotide sequence that hybridizes under stringent conditions to at least about 12 or 15, preferably about 20 or 25, more preferably about 30, 35, 40, 45, 50, 55, 60, 65, 75, 80, 85, 90, 95 or 100 consecutive polynucleotides of a sense sequence of a full-length Ccdc80 polynucleotide sequence or of a naturally occurring allelic variant or mutant of said full-length sequence. In another embodiment, a polynucleotide comprises a polynucleotide sequence which is at least about 100, 200, 300, 400, 500, 600, or 700 nucleotides in length and hybridizes under stringent hybridization conditions to a polynucleotide sequence of a full-length Ccdc80 polynucleotide sequence or a complement thereof.

A nucleic acid fragment encoding a “biologically active portion of a Ccdc80 protein” can be prepared by isolating a portion of a full-length Ccdc80 polynucleotide sequence which encodes a polypeptide having a Ccdc80 biological activity (e.g., activating osteoblasts and/or enhancing bone remodeling), expressing the encoded portion of a Ccdc80 protein (e.g., by recombinant expression in vitro), and assessing the activity of the encoded portion of the Ccdc80 protein.

### III. ISOLATED CCDC80 PROTEINS AND FRAGMENTS THEREOF

Native Ccdc80 proteins can be isolated from cells or tissue sources by an appropriate purification scheme using standard protein purification techniques. In another embodiment, Ccdc80 proteins are produced by recombinant DNA techniques. Alternative to recombinant expression, a Ccdc80 protein or polypeptide can be synthesized chemically using standard peptide synthesis techniques. It will be understood that in discussing the uses of Ccdc80 proteins, e.g., human, mouse, rat, or chicken Ccdc80 (GenBank® accession numbers NM_{—}199511, NM_{—}026439, NM_{—}022543, NM_{—}204431), that fragments of such proteins that are not full-length Ccdc80 polypeptides as well as full-length Ccdc80 proteins can be used.

In a preferred embodiment, a Ccdc80 protein comprises the amino acid sequence of any of the aforementioned GenBank® sequences or a portion thereof. In other embodiments, a Ccdc80 protein has at least 65%, at least 70% amino acid identity, at least 80% amino acid identity, at least 85% amino acid identity, at least 90% amino acid identity, or at least 95% amino acid identity with the amino acid sequence shown in of any of the aforementioned GenBank® sequences portion thereof. Preferred portions of Ccdc80 polypeptide molecules are biologically active, for example, a portion of the Ccdc80 polypeptide having the ability to enhance osteoblast activity and/or bone remodeling.

Biologically active portions of a Ccdc80 protein include peptides comprising amino acid sequences sufficiently homologous to or derived from the amino acid sequence of the Ccdc80 protein, which include less amino acids than the full-length Ccdc80 proteins, and exhibit at least one activity of a Ccdc80 protein.

The invention also provides Ccdc80 chimeric or fusion proteins. Ccdc80 chimeric or fusion proteins or polynucleotides encoding Ccdc80 chimeric or fusion proteins may be employed in the methods of the present invention. In one embodiment, the fusion protein is a GST-Ccdc80 member fusion protein in which the Ccdc80 member sequences are fused to the C-terminus of the GST sequences. In another embodiment, the fusion protein is a Ccdc80-HA fusion protein in which the Ccdc80 member polynucleotide sequence is inserted in a vector such as pCEP4-HA vector (Herrscher R F et al., Genes Dev. 9:3067-82 (1995)) such that the Ccdc80 member sequences are fused in frame to an influenza hemagglutinin epitope tag. In a further embodiment, the fusion protein may be an Fc-fusion protein. For example, a useful Fc fusion protein may be a chimeric protein consisting of Ccdc80 fused to the Fc region of an immunoglobulin G (IgG). The fusion can occur at either the N- or C-terminus of the Fc region. The Fc fusion protein may be expressed in cells using an expression plasmid. The resulting Fc fusion protein can be secreted into culture medium. For example, in some embodiments, the Fc region of immunoglobulin may be used as the N-terminal fusion partner, which can direct the cellular processes into expressing and secreting high levels of many different types of proteins, including, but not limited to, secreted proteins, such as Ccdc80.

Such fusion proteins can facilitate the purification of a recombinant Ccdc80 member. For example, with respect to Fc-fusion proteins, the Fc region provides for easy detection and purification. In particular, Fc-fusion proteins can be purified in a single-step using protein A or protein G affinity chromatography according to methods well known in the art. Protein A and protein G bind specifically to the Fc region of IgG. With respect to Fc-fusion proteins, the Fc region also provides for improved pharmaceutical properties (e.g., altered half-life and effector functions), and may be used as a therapeutic.

Fusion proteins and peptides produced by recombinant techniques may be secreted and isolated from a mixture of cells and medium containing the protein or peptide. Alternatively, the protein or peptide may be retained cytoplasmically and the cells harvested, lysed, and the protein isolated. A cell culture typically includes host cells, media, and other byproducts. Suitable media for cell culture are well known in the art. Protein and peptides can be isolated from cell culture media, host cells, or both using techniques known in the art for purifying proteins and peptides. Techniques for transfecting host cells and purifying proteins and peptides are known in the art.

In one embodiment, a Ccdc80 fusion protein is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al., John Wiley & Sons: 1992). Moreover, many expression vectors are commercially available that already encode a fusion moiety (e.g., a GST polypeptide or an HA epitope tag). A Ccdc80-encoding nucleic acid can be cloned into such an expression vector such that the fusion moiety is linked in-frame to the Ccdc80 protein.

In another embodiment, the fusion protein is a Ccdc80 protein containing a heterologous signal sequence at its N-terminus. In certain host cells (e.g., mammalian host cells), expression and/or secretion of Ccdc80 can be increased through use of a heterologous signal sequence. The Ccdc80 fusion proteins of the invention can be incorporated into pharmaceutical compositions and administered to a subject in vivo. Ccdc80 fusion proteins may be useful therapeutically for the treatment of bone disorders where bone formation lags behind bone resorption (such as, for example, osteoporosis).

The present invention also pertains to variants of Ccdc80 proteins which function as Ccdc80 agonists (mimetics). Variants of Ccdc80 proteins can be generated by mutagenesis, for example, discrete point mutation or truncation of a Ccdc80 protein. An agonist of a Ccdc80 protein can retain substantially the same, or a subset, of the biological activities of the naturally occurring form of a Ccdc80 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of a Ccdc80 protein.

In one embodiment, the invention pertains to derivatives of Ccdc80, which may be formed by modifying at least one amino acid residue of Ccdc80 by oxidation, reduction, or other derivatization processes known in the art.

In one embodiment, variants of a Ccdc80 protein which function as Ccdc80 agonists (mimetics) can be identified by screening combinatorial libraries of mutants, for example, truncation mutants, of a Ccdc80 protein for Ccdc80 protein agonist activity. In one embodiment, a variegated library of Ccdc80 variants is generated by combinatorial mutagenesis at the nucleic acid level and is encoded by a variegated gene library. A variegated library of Ccdc80 variants can be produced by, for example, enzymatically ligating a mixture of synthetic oligonucleotides into gene sequences such that a degenerate set of potential Ccdc80 sequences is expressible as individual polypeptides, or alternatively, as a set of larger fusion proteins (e.g., for phage display) containing the set of Ccdc80 sequences therein. There are a variety of methods which can be used to produce libraries of potential Ccdc80 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential Ccdc80 sequences. Methods for synthesizing degenerate oligonucleotides are known in the art (see, e.g., Narang S A, Tetrahedron 39:3-22 (1983); Itakura K et al., Annu. Rev. Biochem. 53:323-56 (1984); Itakura K et al., Science 198:1056-63 (1977); Ike Y et al., Nucleic Acids Res. 11:477-88 (1983)).

In addition, libraries of fragments of a Ccdc80 protein coding sequence can be used to generate a variegated population of Ccdc80 fragments for screening and subsequent selection of variants of a Ccdc80 protein. In one embodiment, a library of coding sequence fragments can be generated by treating a double stranded PCR fragment of a Ccdc80 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with SI nuclease, and ligating the resulting fragment library into an expression vector. By this method, an expression library can be derived which encodes N-terminal, C-terminal, and internal fragments of various sizes of a Ccdc80 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of Ccdc80 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify Ccdc80 variants (Arkin A P and Youvan D C, Proc. Natl. Acad. Sci. USA 89:7811-15 (1992); Delgrave S et al., Protein Eng. 6:327-31 (1993)).

In one embodiment, cell based assays can be exploited to analyze a variegated Ccdc80 library. For example, a library of expression vectors can be transfected into a cell line which ordinarily synthesizes and secretes Ccdc80. The transfected cells are then cultured such that Ccdc80 and a particular mutant Ccdc80 are secreted and the effect of expression of the mutant on Ccdc80 activity in cell supernatants can be detected, for example, by any of a number of enzymatic assays. Plasmid DNA can then be recovered from the cells which score for inhibition, or alternatively, potentiation of Ccdc80 activity, and the individual clones further characterized.

In addition to Ccdc80 polypeptides consisting only of naturally-occurring amino acids, Ccdc80 peptidomimetics are also useful. Peptide analogs are commonly used in the pharmaceutical industry as non-peptide drugs with properties analogous to those of the template peptide. These types of non-peptide compound are termed “peptide mimetics” or “peptidomimetics” (Fauchere J, Adv. Drug Res. 15:29 (1986); Veber D F and Freidinger R M, Trends Neurosci. 8:392-96 (1985); Evans B E et al., J. Med. Chem. 30:1229-39 (1987)) and are usually developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), such as human Ccdc80, but have one or more peptide linkages optionally replaced by a linkage selected from the group consisting of: —CH₂NH—, —CH₂S—, —CH₂CH₂—, —CH═CH— (cis and trans), —COCH₂—, —CH(OH)CH₂—, and —CH₂SO—, by methods known in the art and further described in the following references: Spatola A F in “Chemistry and Biochemistry of Amino Acids, Peptides, and Proteins,” B. Weinstein, ed., Marcel Dekker, New York, p. 267 (1983); Spatola, A F, Vega Data (March 1983), Vol. 1, Issue 3, “Peptide Backbone Modifications” (general review); Morley J S, Trends Pharmcol. Sci. 1:463-68 (1980) (general review); Hudson D et al., Int. J. Pept. Prot. Res. 14:177-85 (1979) (—CH₂NH—, CH₂CH₂—); Spatola A F et al., Life Sci. 38:1243-49 (1986) (—CH₂S—); Hann M M, J. Chem. Soc. Perkin Trans. 1, 307-314 (1982) (—CH═CH—, cis and trans); Almquist R G et al., J. Med. Chem. 23:1392-98 (1980) (—COCH₂—); Jennings-White C et al., Tetrahedron Lett. 23:2533-34 (1982) (—COCH₂—); EP 0 045 665 (—CH(OH)CH₂—); Holladay M W et al., Tetrahedron Lett., 24:4401-04 (1983) (—C(OH)CH₂—); Hruby V J, Life Sci. 31:189-99 (1982) (—CH₂S—). A particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivatization (e.g., labeling) of peptidomimetics should not substantially interfere with the desired biological or pharmacological activity of the peptidomimetic.

Systematic substitution of one or more amino acids of a Ccdc80 amino acid sequence with a D-amino acid of the same type (e.g., D-lysine in place of L-lysine) may be used to generate more stable peptides. In addition, constrained peptides comprising a Ccdc80 amino acid sequence or a substantially identical sequence variation may be generated by methods known in the art (Rizo J and Gierasch L M, Ann. Rev. Biochem. 61:387-416 (1992)); for example, by adding internal cysteine residues capable of forming intramolecular disulfide bridges which cyclize the peptide.

Amino acid sequences of Ccdc80 polypeptides will enable those of skill in the art to produce polypeptides corresponding to Ccdc80 peptide sequences and sequence variants thereof. Such polypeptides may be produced in prokaryotic or eukaryotic host cells by expression of polynucleotides encoding a Ccdc80 peptide sequence, frequently as part of a larger polypeptide. Alternatively, such peptides may be synthesized by chemical methods. Methods for expression of heterologous proteins in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Gutte B and Merrifield R B, J. Am. Chem. Soc. 91:501-02 (1969); Chaiken I M, CRC Crit. Rev. Biochem. 11:255-301 (1981); Kaiser E T et al., Science 243:187-92 (1989); Merrifield B, Science 232:341-47 (1986); Kent S B H, Ann. Rev. Biochem. 57:957-89 (1988); Offord, R. E. (1980) Semisynthetic Proteins, Wiley Publishing.

Peptides can be produced, for example, by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, may be incorporated into various embodiments of the invention. Certain amino-terminal and/or carboxy-terminal modifications and/or peptide extensions to the core sequence can provide advantageous physical, chemical, biochemical, and pharmacological properties such as, for example, enhanced stability, increased potency and/or efficacy, resistance to serum proteases, desirable pharmacokinetic properties, and others. Peptides may be used therapeutically to treat disease.

An isolated Ccdc80 protein, or a portion or fragment thereof, can also be used as an immunogen to generate antibodies that bind Ccdc80 using standard techniques for polyclonal and monoclonal antibody preparation. A full-length Ccdc80 protein can be used or, alternatively, the invention provides antigenic peptide fragments of Ccdc80 for use as immunogens. The antigenic peptide of Ccdc80 comprises at least 8 amino acid residues and encompasses an epitope of Ccdc80 such that an antibody raised against the peptide forms a specific immune complex with Ccdc80. In other embodiments, the antigenic peptide comprises at least 10 amino acid residues, at least 15 amino acid residues, at least 20 amino acid residues, or at least 30 amino acid residues.

In one embodiment, epitopes encompassed by the antigenic peptide are regions of a Ccdc80 polypeptide that are located on the surface of the protein, for example, hydrophilic regions, and that are unique to a Ccdc80 polypeptide. In one embodiment, such epitopes can be specific for a Ccdc80 protein from one species, such as mouse or human (i.e., an antigenic peptide that spans a region of a Ccdc80 polypeptide that is not conserved across species is used as immunogen; such non-conserved residues can be determined using an alignment program such as that described herein). A standard hydrophobicity analysis of the protein can be performed to identify hydrophilic regions.

A Ccdc80 immunogen typically is used to prepare antibodies by immunizing a suitable subject (e.g., rabbit, goat, mouse, or other mammal) with the immunogen. An appropriate immunogenic preparation can contain, for example, a recombinantly expressed Ccdc80 protein or a chemically synthesized Ccdc80 peptide. The preparation can further include an adjuvant, such as Freund's complete or incomplete adjuvant, or similar immunostimulatory agent. Immunization of a suitable subject with an immunogenic Ccdc80 preparation induces a polyclonal anti-Ccdc80 antibody response.

### IV. ANTIBODIES

Accordingly, another aspect pertains to the use of anti-Ccdc80 antibodies. Polyclonal anti-Ccdc80 antibodies can be prepared as described above by immunizing a suitable subject with a Ccdc80 immunogen. The anti-Ccdc80 antibody titer in the immunized subject can be monitored over time by standard techniques, such as with an enzyme linked immunosorbent assay (ELISA) using immobilized Ccdc80 polypeptide. If desired, the antibody molecules directed against a Ccdc80 polypeptide can be isolated from the mammal (e.g., from the blood) and further purified by well known techniques, such as protein A chromatography to obtain the IgG fraction. At an appropriate time after immunization, for example, when the anti-Ccdc80 antibody titers are highest, antibody-producing cells can be obtained from the subject and used to prepare monoclonal antibodies by standard techniques, such as the hybridoma technique originally described by Kohler G and Milstein C, Nature 256:495-97 (1975) (see also, Brown J P et al., J. Immunol. 127:539-46 (1981); Brown J P et al., J. Biol. Chem. 255:4980-83 (1980); Yeh M Y et al., Proc. Natl. Acad. Sci. USA 76:2927-31 (1979); Yeh M Y et al., Int. J. Cancer 29:269-75 (1982)), the more recent human B cell hybridoma technique (Kozbor D and Roder J C, Immunol. Today 4:72-79 (1983)), the EBV-hybridoma technique (Cole et al. (1985), Monoclonal Antibodies and Cancer Therapy, Alan R. Liss, Inc., pp. 77-96), or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); Lerner E A, Yale J. Biol. Med., 54:387-402 (1981); Gefter M L et al., Somatic Cell Genet. 3:231-36 (1977)). Briefly, an immortal cell line (typically a myeloma) is fused to lymphocytes (typically splenocytes) from a mammal immunized with a Ccdc80 immunogen as described above, and the culture supernatants of the resulting hybridoma cells are screened to identify a hybridoma producing a monoclonal antibody that binds specifically to a Ccdc80 polypeptide.

An anti-Ccdc80 antibody (e.g., monoclonal antibody) can be used to isolate a Ccdc80 polypeptide by standard techniques, such as affinity chromatography or immunoprecipitation. Anti-Ccdc80 antibodies can facilitate the purification of natural Ccdc80 polypeptides from cells and of recombinantly produced Ccdc80 polypeptides expressed in host cells. Moreover, an anti-Ccdc80 antibody can be used to detect a Ccdc80 protein (e.g., in a cellular lysate or cell supernatant). Detection may be facilitated by coupling (i.e., physically linking) the antibody to a detectable substance. Accordingly, in one embodiment, an anti-Ccdc80 antibody of the invention is labeled with a detectable substance. Examples of detectable substances include various enzymes, prosthetic groups, fluorescent materials, luminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, or phycoerythrin; an example of a luminescent material includes luminol; and examples of suitable radioactive material include ¹²⁵I, ¹³¹I, ³⁵S, or ³H.

In some embodiments, antibodies that recognize extracellular Ccdc80 may used to inhibit Ccdc80 protein activity. For example, to produce soluble (secreted) Ccdc80 protein, a Ccdc80-Fc fusion protein may be generated by PCR, sequenced, and cloned into an expression vector, and then transfected into cells, such as CHO cells. The soluble Ccdc80-Fc fusion protein is secreted into the culture medium by the transfected cells, and then purified from the culture medium by using, for example, protein A chromatography according to methods well known in the art. Subjects, such as rabbits, rats or mice, may then be immunized with purified Ccdc80-Fc fusion protein mixed with an adjuvant. The anti-Ccdc80 antibody titer in the sera of the immunized subject(s) can be monitored over time by standard techniques, such as with an enzyme linked immunosorbent assay (ELISA) using an immobilized Ccdc80 polypeptide.

Polyclonal antibody molecules directed against the extracellular Ccdc80 polypeptide can be isolated from the immunized mammal (e.g., from the sera) and further purified by well known techniques, such as protein A chromatography to obtain the IgG fraction. Alternatively, an anti-Ccdc80 monoclonal antibody may be generated. For example, cells from the spleens of the immunized subjects having the highest anti-Ccdc80 specific response may be used to prepare monoclonal antibodies by standard techniques, such as the hybridoma technique originally described by Kohler G and Milstein C, Nature 256:495-97 (1975). Polyclonal or monoclonal antibodies that recognize extracellular Ccdc80, or an extracellular domain thereof, may be used to inhibit the functional activity of extracellular Ccdc80 protein.

In one embodiment, a recombinant expression vector is prepared which encodes the antibody chains in a form such that, upon introduction of the vector into a cell, the antibody chains are expressed by the cell as a functional antibody. For inhibition of secreted Ccdc80 activity, an antibody that specifically binds to Ccdc80 preferably recognizes extracellular Ccdc80, and is secreted from the cell. For example, an expression plasmid may be used to facilitate the generation of an Fc-fusion protein where the fusion protein is a chimeric protein consisting of the Fab region of the anti-Ccdc80 antibody fused to the Fc region of an immunoglobulin G (IgG). The Fc region provides a handle for detection of the antibody. To inhibit Ccdc80 activity, an expression vector encoding anti-Ccdc80 antibody may introduced into a cell by standard transfection methods, as discussed herein. As described in further detail herein, inhibition of Ccdc80 activity would be useful to treat bone disorders characterized by an excessive production of Ccdc80, for example.

### V. RECOMBINANT EXPRESSION VECTORS AND HOST CELLS

Recombinant expression vectors can comprise a nucleic acid in a form suitable for expression of the nucleic acid in a host cell, which means that the recombinant expression vectors include one or more regulatory sequences, selected on the basis of the host cells to be used for expression, that are operably linked to the nucleic acid sequence to be expressed. The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., Ccdc80 proteins, mutant forms of Ccdc80 proteins, fusion proteins, and the like).

Recombinant expression vectors can be designed for expression of proteins or protein fragments in prokaryotic or eukaryotic cells. For example, Ccdc80 proteins can be expressed in bacterial cells such as E. coli, insect cells (using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, and enterokinase. Typical fusion expression vectors include, for example, pGEX (Pharmacia Biotech Inc.; Smith D B and Johnson K S, Gene 67:31-40 (1988)) and pMAL (New England Biolabs, Beverly, Mass.) which fuse glutathione S-transferase (GST) or maltose E binding protein, respectively, to the target recombinant protein.

Examples of suitable inducible non-fusion E. coli expression vectors include pTrc (Amann E et al., Gene 69:301-15 (1988)) and pET 11d (Studier et al., Gene Expression Technology: Methods in Enzymology 185, Academic Press, San Diego, Calif. (1990) pp. 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman S, Gene Expression Technology: Methods in Enzymology 185, Academic Press, San Diego, Calif. (1990) pp. 119-28). Another strategy is to alter the nucleic acid sequence of the nucleic acid to be inserted into an expression vector so that the individual codons for each amino acid are those preferentially utilized in E. coli (Wada K et al., Nucleic Acids Res. 20(Suppl.):2111-18 (1992)). Such alteration of nucleic acid sequences of the invention can be carried out by standard DNA synthesis techniques.

In another embodiment, the expression vector is a yeast expression vector. Examples of vectors for expression in yeast S. cerevisiae include pYepSec1 (Baldari C et al., EMBO J. 6:229-34 (1987)), pMFa (Kurjan J and Herskowitz I, Cell 30:933-43 (1982)), pJRY88 (Schultz L D et al., Gene 54:113-23 (1987)), pYES2 (Invitrogen Corp., San Diego, Calif.), and picZ (Invitrogen Corp).

Alternatively, proteins or polypeptides can be expressed in insect cells using baculovirus expression vectors. Baculovirus vectors available for expression of proteins in cultured insect cells (e.g., Sf 9 cells) include the pAc series (Smith G E et al., Mol. Cell. Biol. 3:2156-65 (1983)) and the pVL series (Lucklow V A and Summers M D, Virology 170:31-39 (1989)).

In yet another embodiment, nucleic acids are expressed in mammalian cells using a mammalian expression vector. Examples of mammalian expression vectors include pCDM8 (Seed B, Nature 329:840-41 (1987)) and pMT2PC (Kaufman R J et al., EMBO J. 6:187-95 (1987)). When used in mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see chapters 16 and 17 of Sambrook, J., Fritsh, E. F., and Maniatis, T. Molecular Cloning: A Laboratory Manual, 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert C A et al., Genes Dev. 1:268-77 (1987)), lymphoid-specific promoters (Calame K and Eaton S, Adv. Immunol. 43:235-75 (1988)), in particular promoters of T cell receptors (Winoto A and Baltimore D, EMBO J. 8:729-33 (1989)) and immunoglobulins (Banerji J et al., Cell 33:729-40 (1983); Queen C and Baltimore D, Cell 33:741-48 (1983)), neuron-specific promoters (e.g., the neurofilament promoter; Byrne G W and Ruddle F H, Proc. Natl. Acad. Sci. USA 86:5473-77 (1989)), pancreas-specific promoters (Edlund T et al., Science 230:912-16 (1985)), and mammary gland-specific promoters (e.g., milk whey promoter; U.S. Pat. No. 4,873,316 and EP 0 264 166). Developmentally-regulated promoters are also encompassed, for example the murine hox promoters (Kessel M and Gruss P, Science 249:374-79 (1990)) and the α-fetoprotein promoter (Camper S A and Tilghman S M, Genes Dev. 3:537-46 (1989)).

Moreover, inducible regulatory systems for use in mammalian cells are known in the art, for example systems in which gene expression is regulated by heavy metal ions (see e.g., Mayo K E et al., Cell 29:99-108 (1982); Brinster R L et al., Nature 296:39-42 (1982); Searle P F et al., Mol. Cell. Biol. 5:1480-89 (1985)), heat shock (see e.g., Nouer L et al. (1991) in Heat Shock Response, ed. Nouer L, CRC, Boca Raton, Fla., pp. 167-220), hormones (see e.g., Lee F et al., Nature 294:228-32 (1981); Hynes N E et al., Proc. Natl. Acad. Sci. USA 78:2038-42 (1981); Klock G et al., Nature 329:734-36 (1987); Israel D I and Kaufman R J, Nucleic Acids Res. 17:2589-2604 (1989); WO 93/23431), FK506-related molecules (see e.g., WO 94/18317) or tetracyclines (Gossen M and Bujard H, Proc. Natl. Acad. Sci. USA 89:5547-51 (1992); Gossen M et al., Science 268:1766-69 (1995); WO 94/29442; WO 96/01313). Accordingly, in another embodiment, the invention provides a recombinant expression vector in which a DNA is operably linked to an inducible eukaryotic promoter, thereby allowing for inducible expression of a protein in eukaryotic cells.

Also known in the art are methods for expressing endogenous proteins using one-arm homologous recombination (see, e.g., U.S. Published Patent Application No. 2005/0003367; Zeh et al., Assay Drug Dev. Technol. 1:755-65 (2003); Qureshi et al., Assay Drug Dev. Technol. 1:767-76 (2003)). Briefly, an isolated genomic construct comprising a promoter operably linked to a targeting sequence is introducing into a homogeneous population of cells (such as, for example, a homogeneous population of a human cell line). The promoter is heterologous to the target gene. Following recombination, the promoter controls transcription of an mRNA that encodes a polypeptide. The population of cells is then incubated under conditions which cause expression of the polypeptide.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including, for example, calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual, 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, and methotrexate. A nucleic acid molecule encoding a selectable marker can be introduced into a host cell on the same vector as that encoding a protein or can be introduced on a separate vector. Cells stably transfected with the introduced nucleic acid molecule can be identified by drug selection (e.g., cells that have incorporated the selectable marker gene will survive, while the other cells die).

In the case of, for example, HepG2 (e.g., stably transfected with mammalian expression vectors (e.g., pSmed2) encoding, e.g., human Ccdc80; infected with an adenovirus (e.g., pAdori backbone vector) encoding, e.g., human Ccdc80), HEK293T e.g., stably transfected with mammalian expression vectors (e.g., pSmed2) encoding, e.g., human Ccdc80), 3T3-L1 (e.g., infected with an adenovirus (e.g., pAdori backbone vector; pAdEasy backbone vector) encoding, e.g., human or mouse Ccdc80), or C2C12 cells (e.g., infected with an adenovirus (e.g., pAdori backbone vector) encoding, e.g., human Ccdc80) which are stably transfected with Ccdc80, such lines can be made so that the Ccdc80 gene is inducible by methods well-known to those skilled in the art.

In one embodiment, a host cell of invention into which a recombinant expression vector encoding a Ccdc80 protein has been introduced is cultured in a suitable medium such that a Ccdc80 protein is produced. In another embodiment, a Ccdc80 protein is isolated from the medium or the host cell.

### VI. USES AND METHODS OF THE INVENTION

The Ccdc80 modulators described herein can be used in one or more of the following methods: a) methods of treatment, preferably in bone cells; b) screening assays; c) predictive medicine (e.g., diagnostic assays, prognostic assays, monitoring clinical trials, or pharmacogenetics); and d) cosmetic treatment methods. With respect to cosmetic treatment, an agent that stimulates expression or activity of the Ccdc80 gene or Ccdc80 protein may be useful in the cosmetic repair of bone defects and deficiencies. For example, a Ccdc80 stimulatory agent may be used in the cosmetic repair of congenital, trauma-induced or surgical resection of bone (for instance, for cancer treatment).

The isolated nucleic acid molecules of the invention can be used, for example, to express Ccdc80 protein (e.g., via a recombinant expression vector in a host cell in gene therapy applications) and to modulate Ccdc80 activity, as described further below. In addition, the Ccdc80 proteins can be used to screen for naturally occurring Ccdc80 binding proteins, to screen for drugs or compounds which modulate Ccdc80 activity, as well as to treat disorders that would benefit from modulation of Ccdc80, for example, characterized by insufficient or excessive production of Ccdc80 protein or production of Ccdc80 protein forms which have decreased or aberrant activity compared to Ccdc80 wild type protein. As described herein, there are many conditions that are characterized by the need to enhance bone formation. For example, in the case of bone fractures, it would be desirable to stimulate bone growth and to hasten and complete bone repair. As another example, in the case of diseases that result in bone loss, it would be desirable to stimulate bone growth.

### A. Methods of Modulating Ccdc80

According to one modulatory method, Ccdc80 activity is stimulated in a cell by contacting the cell with a stimulatory agent. Examples of such stimulatory agents include active Ccdc80 protein, fragments thereof, Ccdc80 agonists (e.g., a Ccdc80 peptidomimetic), and nucleic acid molecules encoding Ccdc80 or a fragment thereof that are introduced into the cell to increase Ccdc80 activity in the cell. To express a Ccdc80 protein in a cell, typically a Ccdc80 cDNA is first introduced into a recombinant expression vector using standard molecular biology techniques, as described herein. A Ccdc80 cDNA can be obtained, for example, by amplification using the PCR or by screening an appropriate cDNA library as described herein. Following isolation or amplification of Ccdc80 cDNA, the DNA fragment is introduced into an expression vector and transfected into target cells by standard methods, as described herein. Other stimulatory agents that can be used to stimulate the activity and/or expression of a Ccdc80 protein are chemical compounds that stimulate Ccdc80 activity and/or expression in cells, such as compounds that enhance Ccdc80 activation of osteoblasts and/or bone remodeling. Such compounds can be identified using screening assays that select for such compounds, as described in detail herein.

Modulatory methods can be performed in vitro (e.g., by culturing the cell with the agent or by introducing the agent into cells in culture) or, alternatively, in vivo (e.g., by administering the agent to a subject or by introducing the agent into cells of a subject, such as by gene therapy). For practicing a modulatory method in vitro, cells can be obtained from a subject by standard methods and incubated (i.e., cultured) in vitro with a modulatory agent to modulate Ccdc80 activity in the cells.

For agents that comprise nucleic acids (including recombinant expression vectors encoding Ccdc80 protein), the agents can be introduced into cells of the subject using methods known in the art for introducing nucleic acid (e.g., DNA) into cells in vivo. Examples of such methods encompass both non-viral and viral methods, including:

Direct Injection: Naked DNA can be introduced into cells in vivo by directly injecting the DNA into the cells (see, e.g., Acsadi G et al., Nature 332:815-18 (1991); Wolff J A et al., Science 247:1465-68 (1990)). For example, a delivery apparatus (e.g., a “gene gun”) for injecting DNA into cells in vivo can be used. Such an apparatus is commercially available (e.g., from Bio-Rad Laboratories, Hercules, Calif.).

Cationic Lipids: Naked DNA can be introduced into cells in vivo by complexing the DNA with cationic lipids or encapsulating the DNA in cationic liposomes. Examples of suitable cationic lipid formulations include N-[-1-(2,3-dioleoyloxy)propyl]N,N,N-triethylammonium chloride (DOTMA) and a 1:1 molar ratio of 1,2-dimyristyloxy-propyl-3-dimethylhydroxyethylammonium bromide (DMRIE) and dioleoyl phosphatidylethanolamine (DOPE) (see e.g., Logan J J et al., Gene Ther. 2:38-49 (1995); San H et al., Hum. Gene Ther. 4:781-88 (1993)).

Receptor-Mediated DNA Uptake: Naked DNA can also be introduced into cells in vivo by complexing the DNA to a cation, such as polylysine, which is coupled to a ligand for a cell-surface receptor (see, e.g., Wu G Y and Wu C H, J. Biol. Chem. 263:14621-24 (1988); Wilson J M et al., J. Biol. Chem. 267:963-67 (1992); and U.S. Pat. No. 5,166,320). Binding of the DNA-ligand complex to the receptor facilitates uptake of the DNA by receptor-mediated endocytosis. A DNA-ligand complex linked to adenovirus capsids which naturally disrupt endosomes, thereby releasing material into the cytoplasm can be used to avoid degradation of the complex by intracellular lysosomes (see, e.g., Curiel D T et al., Proc. Natl. Acad. Sci. USA 88:8850-54 (1991); Cristiano R J et al., Proc. Natl. Acad. Sci. USA 90:2122-26 (1993)).

Retroviruses: Defective retroviruses are well characterized for use in gene transfer for gene therapy purposes (for a review, see Miller A D, Blood 76:271-78 (1990)). A recombinant retrovirus can be constructed having a nucleotide sequence of interest incorporated into the retroviral genome. Additionally, portions of the retroviral genome can be removed to render the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel F M et al. (eds.) Greene Publishing Associates, (1989), Sections 9.10-9.14 and other standard laboratory manuals. Examples of suitable retroviruses include pLJ, pZIP, pWE, and pEM which are well known to those skilled in the art. Examples of suitable packaging virus lines include ψCrip, ψCre, ψ2 and ψAm. Retroviruses have been used to introduce a variety of genes into many different cell types, including epithelial cells, endothelial cells, lymphocytes, myoblasts, hepatocytes, bone marrow cells, in vitro and/or in vivo (see, e.g., Eglitis M A et al., Science 230:1395-98 (1985); Danos O and Mulligan R C, Proc. Natl. Acad. Sci. USA 85:6460-64 (1988); Wilson J M et al., Proc. Natl. Acad. Sci. USA 85:3014-18 (1988); Armentano D et al., Proc. Natl. Acad. Sci. USA 87:6141-45 (1990); Huber B E et al., Proc. Natl. Acad. Sci. USA 88:8039-43 (1991); Ferry N et al., Proc. Natl. Acad. Sci. USA 88:8377-81 (1991); Chowdhury J R et al., Science 254:1802-05 (1991); van Beusechem V W et al., Proc. Natl. Acad. Sci. USA 89:7640-44 (1992); Kay M A et al., Hum. Gene Ther. 3:641-47 (1992); Dai Y et al., Proc. Natl. Acad. Sci. USA 89:10892-95 (1992); Hwu P et al., J. Immunol. 150:4104-15 (1993); U.S. Pat. No. 4,868,116; U.S. Pat. No. 4,980,286; WO 89/07136; WO 89/02468; WO 89/05345; and WO 92/07573). Retroviral vectors require target cell division in order for the retroviral genome (and foreign nucleic acid inserted into it) to be integrated into the host genome to stably introduce nucleic acid into the cell. Thus, it may be necessary to stimulate replication of the target cell.

Adenoviruses: The genome of an adenovirus can be manipulated such that it encodes and expresses a gene product of interest but is inactivated in terms of its ability to replicate in a normal lytic viral life cycle (see, e.g., Berkner K L, Biotechniques 6:616-29 (1988); Rosenfeld M A et al., Science 252:431-34 (1991); and Rosenfeld M A et al., Cell 68:143-55 (1992)). Suitable adenoviral vectors derived from the adenovirus strain Ad type 5 d1324 or other strains of adenovirus (e.g., Ad2, Ad3, Ad7, etc.) are well known to those skilled in the art. Recombinant adenoviruses are advantageous in that they do not require dividing cells to be effective gene delivery vehicles and can be used to infect a wide variety of cell types, including airway epithelium (Rosenfeld M A et al., Cell 68:143-55 (1992)), endothelial cells (Lemarchand P et al., Proc. Natl. Acad. Sci. USA 89:6482-86 (1992)), hepatocytes (Herz J and Gerard R D, Proc. Natl. Acad. Sci. USA 90:2812-16 (1993)), and muscle cells (Quantin B et al., Proc. Natl. Acad. Sci. USA 89:2581-84 (1992)). Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Moreover, the carrying capacity of the adenoviral genome for foreign DNA is large (up to 8 kilobases) relative to other gene delivery vectors (Berkner K L et al., supra; Haj-Ahmad Y and Graham F L, J. Virol. 57:267-74 (1986)). Most replication-defective adenoviral vectors currently in use are deleted for all or parts of the viral E1 and E3 genes but retain as much as 80% of the adenoviral genetic material.

Adeno-Associated Viruses: Adeno-associated virus (AAV) is a naturally occurring defective virus that requires another virus, such as an adenovirus or a herpes virus, as a helper virus for efficient replication and a productive life cycle (for a review, see Muzyczka N, Curr. Top. Microbiol. Immunol. 158:97-129 (1992)). It is also one of the few viruses that may integrate its DNA into non-dividing cells, and exhibits a high frequency of stable integration (see, e.g., Flotte T R et al., Am. J. Respir. Cell. Mol. Biol. 7:349-56 (1992); Samulski R J et al., J. Virol. 63:3822-28 (1989); and McLaughlin S K et al., J. Virol. 62:1963-73 (1988)). Vectors containing as little as 300 base pairs of AAV can be packaged and can integrate. Space for exogenous DNA is limited to about 4.5 kb. An AAV vector such as that described in Tratschin J D et al., Mol. Cell. Biol. 5:3251-60 (1985), can be used to introduce DNA into cells. A variety of nucleic acids have been introduced into different cell types using AAV vectors (see, e.g., Hermonat P L and Muzyczka N, Proc. Natl. Acad. Sci. USA 81:6466-70 (1984); Tratschin J D et al., Mol. Cell. Biol. 4:2072-81 (1985); Wondisford F E et al., Mol. Endocrinol. 2:32-39 (1988); Tratschin J D et al., J. Virol. 51:611-19 (1984); and Flotte T R et al., J. Biol. Chem. 268:3781-90 (1993)).

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection, or reverse transcriptase-polymerase chain reaction (RT-PCR). The gene product can be detected by an appropriate assay, for example by immunological detection of a produced protein, such as with a specific antibody, or by a functional assay to detect a functional activity of the gene product.

### 1. Prophylactic Methods

In one aspect, the invention provides a method for preventing in a subject, a disease or condition that would benefit from modulation of Ccdc80 activity and/or expression, e.g., osteoporosis; metabolic bone diseases such as, for example, rickets, osteomalacia, chronic renal failure and hyperparathyroidism, which result in abnormal or excessive loss of bone mass (osteopenia); Paget's disease; bone fractures, by administering to the subject an agent that modulates Ccdc80 polypeptide expression or at least one Ccdc80 activity, such as a Ccdc80 polypeptide, a Ccdc80 agonist (e.g., a Ccdc80 peptidomimetic), or a Ccdc80 polynucleotide. Subjects at risk for a disease which is caused or contributed to by aberrant Ccdc80 expression or activity can be identified by, for example, any or a combination of diagnostic or prognostic assays as are known to those of ordinary skill in the art. Administration of a prophylactic agent can occur prior to the manifestation of symptoms characteristic of Ccdc80 aberrance, such that a disease or disorder is prevented or, alternatively, delayed in its progression. Depending on the type of Ccdc80 aberrance or condition, for example, a Ccdc80 polypeptide, Ccdc80 polynucleotide, or Ccdc80 agonist agent can be used for treating the subject. The appropriate agent can be determined based on screening assays described herein.

### 2. Therapeutic Methods

Another aspect of the invention pertains to methods of modulating Ccdc80 expression or activity for therapeutic purposes. Accordingly, in an exemplary embodiment, the modulatory method of the invention involves contacting a cell with an agent that modulates one or more of the activities of Ccdc80 protein associated with the cell. An agent that modulates Ccdc80 protein activity can be an agent as described herein, such as a Ccdc80 nucleic acid, a Ccdc80 protein, a Ccdc80 polypeptide, a naturally-occurring target molecule of a Ccdc80 protein (e.g., a Ccdc80 binding protein), a Ccdc80 agonist, a peptidomimetic of a Ccdc80 agonist, or a small molecule. In one embodiment, the agent stimulates one or more Ccdc80 activities. Examples of such stimulatory agents include active Ccdc80 protein, a Ccdc80 peptidomimetic, a Ccdc80 agonist, or a nucleic acid molecule encoding Ccdc80 polypeptide that has been introduced into the cell. These modulatory methods can be performed in vitro (e.g., by culturing the cell with the agent) or, alternatively, in vivo (e.g., by administering the agent to a subject). As such, the present invention provides methods of treating an individual afflicted with a disease or disorder that would benefit from modulation of a Ccdc80 protein, e.g., osteoporosis; metabolic bone diseases such as, for example, rickets, osteomalacia, chronic renal failure and hyperparathyroidism, which result in abnormal or excessive loss of bone mass (osteopenia); Paget's disease; bone fractures, or which is characterized by aberrant expression or activity of a Ccdc80 protein or nucleic acid molecule. In one embodiment, the method involves administering an agent (e.g., an agent identified by a screening assay described herein), or combination of agents that modulates Ccdc80 expression or activity. In another embodiment, the method involves administering a Ccdc80 protein or nucleic acid molecule as therapy to compensate for reduced or aberrant Ccdc80 expression or activity.

Stimulation of Ccdc80 activity is desirable in situations in which Ccdc80 is abnormally downregulated and/or in which increased Ccdc80 activity is likely to have a beneficial effect. Exemplary situations in which Ccdc80 modulation will be desirable are in the treatment of Ccdc80-associated disorders or in the treatment of osteoporosis; metabolic bone diseases such as, for example, rickets, osteomalacia, chronic renal failure and hyperparathyroidism, which result in abnormal or excessive loss of bone mass (osteopenia); Paget's disease; and repair of bone fractures.

### 3. Cosmetic Treatments

Another aspect of the invention pertains to methods of modulating Ccdc80 expression or activity for cosmetic purposes. Accordingly, in an exemplary embodiment, the modulatory method of the invention involves contacting a cell with an agent that modulates one or more of the activities of Ccdc80 protein associated with the cell. An agent that modulates Ccdc80 protein activity can be an agent as described herein, such as a Ccdc80 nucleic acid or a Ccdc80 protein or fragment thereof, a naturally-occurring target molecule of a Ccdc80 protein (e.g., a Ccdc80 binding protein), a Ccdc80 agonist, a Ccdc80 peptidomimetic, a peptidomimetic of a Ccdc80 agonist, or a small molecule. In one embodiment, the agent stimulates one or more Ccdc80 activities. Examples of such stimulatory agents are the same as those described above. These cosmetic treatment methods can be performed in vitro (e.g., by culturing the cell with the agent) or, alternatively, in vivo (e.g., by administering the agent to a subject). As such, the present invention provides methods of cosmetically treating bone defects and deficiencies that would benefit from stimulation of Ccdc80 activity and/or expression. In one embodiment, the method involves administering an agent (e.g., an agent identified by a screening assay described herein), or combination of agents that modulates Ccdc80 expression or activity.

### B. Combination Treatments

Ccdc80 modulators may also be used in conjunction with other therapeutic agent(s). Suitable therapeutic agents for such combinations include for example antiresorptive agents (e.g., 1,25-dihydroxyvitamin D3; analogs thereof, such as 1,25-dihydroxy-19-norvitamin D2, 1α-hydroxyvitamin D2, 1α-hydroxyvitamin D3, 1,25-dihydroxy-22-oxavitamin D3 (oxacalcitril), 1,25-dihydroxy-26,26,26,27,27,27-hexfluorovitamin D3 falecalcitriol), and 1,25-dihydroxydihydrotachysterol; polyphosphonates (e.g., alendronate (Fosamax®); risedronate (Actonel®); etidronate (Didronel®); tiludronate (Skelid®); pamidronate (Aredia®); ibandronate; clodronate, and zoledronate (Zometa®); calcitonin and various derivatives thereof); estrogen or other compounds that modulate estrogen receptor activity (e.g., 17β-estradiol; C-21 progestins such as medroxyprogesterone acetate; a selective estrogen receptor modulator (SERM) (e.g., raloxifene, tamoxifen, tibolone, ospemifene, lasofoxifene, and arzoxifene); an osteo-anabolic agent (e.g., parathyroid hormone and various analogs thereof; androgen (e.g., various forms of testosterone (Andro®, Andryl®, Delatest®, Depotest®, Duratest®, Everone®, Histerone®, Tesanone®, Testex®, Testrin®P.A.) and 17-α methyl testosterone (Android®, Metandren®, Oreton® Methyl, Virilon®). Other types of androgens include nandrolone decanoate (e.g., Androlone®, Duraboline®, Hybolin® Improved, Neo-Durabolic), norethisterone acetate, fluoxymesterone (Halotestin®); a fluoride (e.g., sodium fluoride, monofluorophosphate); strontium (e.g., strontium ranelate, available under the tradename Protelos®); growth hormone (GH) (e.g., human growth hormone (hGH), and naturally occurring derivatives and engineered variants of hGH produced by recombinant methods); insulin like growth factor (IGH-I); and/or calcium (e.g., calcium carbonate and calcium chelates, such as calcium citrate, calcium citrate malate, calcium lactate, calcium gluconate, calcium aspartate, and calcium orotate).

In some embodiments, a Ccdc80 modulator, such as a Ccdc80 protein or an adenovirus encoding Ccdc80, may be administered in combination with one or more bone morphogenic proteins (BMPs). As described herein, BMP-2 is a well-known osteogenic factor and is used clinically as a bone-inductive agent. Applicants have shown that, in the presence of BMP-2, Ccdc80 overexpression significantly enhanced BMP-mediated transcriptional activity in C2C12 cells above the level seen with BMP-2 alone (Example 4).

The order of administration of a Ccdc80 modulator and an additional therapeutic agent(s) can vary. For example, in some embodiments, a Ccdc80 modulator is administered concurrently with the additional therapeutic agent(s). Alternatively, a Ccdc80 modulator can be administered separately and prior to the additional therapeutic agent(s). In another embodiment, the additional therapeutic agent(s) can administered separately and prior to a Ccdc80 modulator. In many embodiments, these administration regimens will be continued for days, months, or years.

### C. Screening Assays:

The invention provides a method (also referred to herein as a “screening assay”) for identifying modulators, that is, candidate or test compounds or agents (e.g., peptides, peptidomimetics, small molecules, or other drugs) which bind to Ccdc80 proteins, or have a stimulatory or inhibitory effect on, for example, Ccdc80 expression or Ccdc80 activity.

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer, or small molecule libraries of compounds (Lam K S, Anticancer Drug Des. 12:145-67 (1997)).

Examples of methods for the synthesis of molecular libraries can be found in the art, for example in: DeWitt S H et al., Proc. Natl. Acad. Sci. USA 90:6909-13 (1993); Erb E et al., Proc. Natl. Acad. Sci. USA 91:11422-26 (1994); Zuckermann R N et al., J. Med. Chem. 37:2678-85 (1994); Cho C Y et al., Science 261:1303-05 (1993); Carrell T et al., Angew. Chem. Int. Ed. Engl. 33:2059-61 (1994); Carrell T et al., Angew. Chem. Int. Ed. Engl. 33:2061-64 (1994); and Gallop M A et al., J. Med. Chem. 37:1233-51 (1994).

Libraries of compounds may be presented, for example, in solution (e.g., Houghten R A et al., Biotechniques 13:412-21 (1992)), on beads (Lam K S et al., Nature 354:82-84 (1991)), chips (Fodor S P A et al., Nature 364:555-56 (1993)), bacteria (U.S. Pat. No. 5,223,409), spores (U.S. Pat. No. 5,223,409), plasmids (Cull M G et al., Proc. Natl. Acad. Sci. USA 89:1865-69 (1992)), or on phage (Scott J K and Smith G P, Science 249:386-90 (1990); Devlin J J et al., Science 249:404-06 (1990); Cwirla S E et al., Proc. Natl. Acad. Sci. 87:6378-82 (1990); Felici F et al., J. Mol. Biol. 222:301-10 (1991); U.S. Pat. No. 5,223,409).

In many drug screening programs which test libraries of modulating agents and natural extracts, high throughput assays are desirable in order to maximize the number of modulating agents surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test modulating agent. Moreover, the effects of cellular toxicity and/or bioavailability of the test modulating agent can be generally ignored in the in vitro system, the assay instead being focused primarily on the effect of the drug on the molecular target as may be manifest in an alteration of binding affinity with upstream or downstream elements.

Assays can be used to screen for modulating agents, including Ccdc80 homologs, which are either agonists or antagonists of the normal cellular function of the subject Ccdc80 polypeptides. For example, the invention provides a method in which an indicator composition is provided which has a Ccdc80 protein having a Ccdc80 activity. The indicator composition can be contacted with a test compound. The effect of the test compound on Ccdc80 activity, as measured by a change in the indicator composition, can then be determined to thereby identify a compound that modulates the activity of a Ccdc80 protein. A statistically significant change, such as a decrease or increase, in the level of Ccdc80 activity in the presence of the test compound (relative to what is detected in the absence of the test compound) is indicative of the test compound being a Ccdc80 modulating agent. The indicator composition can be, for example, a cell or a cell extract.

The efficacy of the modulating agent can be assessed by generating dose response curves from data obtained using various concentrations of the test modulating agent. Moreover, a control assay can also be performed to provide a baseline for comparison. In the control assay, isolated and purified Ccdc80 protein is added to a composition containing the Ccdc80-binding element, and the formation of a complex is quantitated in the absence of the test modulating agent.

In yet another embodiment, an assay of the present invention is a cell-free assay in which a Ccdc80 protein or biologically active portion thereof is contacted with a test compound and the ability of the test compound to bind to the Ccdc80 protein or biologically active portion thereof is determined. Binding of the test compound to the Ccdc80 protein can be determined either directly or indirectly as described above. In a preferred embodiment, the assay includes contacting the Ccdc80 protein or biologically active portion thereof with a known compound which binds Ccdc80 to form an assay mixture, contacting the assay mixture with a test compound, and determining the ability of the test compound to interact with a Ccdc80 protein, wherein determining the ability of the test compound to interact with a Ccdc80 protein comprises determining the ability of the test compound to preferentially bind to Ccdc80 polypeptide or a biologically active portion thereof as compared to the known compound.

In another embodiment, the assay is a cell-free assay in which a Ccdc80 protein or biologically active portion thereof is contacted with a test compound and the ability of the test compound to modulate the activity of the Ccdc80 protein or biologically active portion thereof is determined. The Ccdc80 protein can be provided as a lysate of cells that express Ccdc80, as a purified or semipurified polypeptide, or as a recombinantly expressed polypeptide. In one embodiment, a cell-free assay system further comprises a cell extract or isolated components of a cell, such as mitochondria. Such cellular components can be isolated using techniques which are known in the art. Preferably, a cell free assay system further comprises at least one target molecule with which Ccdc80 interacts, and the ability of the test compound to modulate the interaction of the Ccdc80 with the target molecule(s) is monitored to thereby identify the test compound as a modulator of Ccdc80. Determining the ability of the test compound to modulate the activity of a Ccdc80 protein can be accomplished, for example, by determining the ability of the Ccdc80 protein to bind to a Ccdc80 target molecule by one of the methods described herein for determining direct binding. Determining the ability of the Ccdc80 protein to bind to a Ccdc80 target molecule can also be accomplished using a technology such as real-time Biomolecular Interaction Analysis (BIA) (see, e.g., Sjolander S and Urbaniczky C, Anal. Chem. 63:2338-45 (1991) and Szabo A et al., Curr. Opin. Struct. Biol. 5:699-705 (1995)).

In yet another embodiment, the cell-free assay involves contacting a Ccdc80 protein or biologically active portion thereof with a known compound which binds the Ccdc80 protein to form an assay mixture, contacting the assay mixture with a test compound, and determining the ability of the test compound to interact with the Ccdc80 protein, wherein determining the ability of the test compound to interact with the Ccdc80 protein comprises determining the ability of the Ccdc80 protein to preferentially bind to or modulate the activity of a Ccdc80 target molecule.

The cell-free assays of the present invention are amenable to use of both soluble and/or membrane-bound forms of proteins (e.g., Ccdc80 proteins or receptors having intracellular domains to which Ccdc80 binds). In the case of cell-free assays in which a membrane-bound form a protein is used, it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl═N,N-dimethyl-3-ammonio-1-propane sulfonate.

Determining the ability of the Ccdc80 protein to bind to or interact with a ligand of a Ccdc80 molecule can be accomplished, for example, by direct binding. In a direct binding assay, the Ccdc80 protein could be coupled with a radioisotope or enzymatic label such that binding of the Ccdc80 protein to a Ccdc80 target molecule can be determined by detecting the labeled Ccdc80 protein in a complex. For example, Ccdc80 molecules, for example, Ccdc80 proteins, can be labeled with, for example, ¹²⁵I, ³⁵S, ¹⁴C, or ³H, either directly or indirectly, and the radioisotope detected by direct counting of radioemmission or by scintillation counting. Alternatively, Ccdc80 molecules can be enzymatically labeled with, for example, horseradish peroxidase, alkaline phosphatase, or luciferase, and the enzymatic label detected by determination of conversion of an appropriate substrate to product.

Typically, it will be desirable to immobilize Ccdc80 or their respective binding proteins to facilitate separation of complexes from uncomplexed forms of one or both of the proteins, as well as to accommodate automation of the assay. Binding of Ccdc80 to an upstream or downstream binding element, in the presence and absence of a candidate agent, can be accomplished in any vessel suitable for containing the reactants. Examples include microtiter plates, test tubes, and micro-centrifuge tubes. In one embodiment, a fusion protein can be provided which adds a domain that allows the protein to be bound to a matrix. For example, glutathione-S-transferase/Ccdc80 (GST/Ccdc80) fusion proteins can be adsorbed onto glutathione sepharose beads (Sigma Chemical, St. Louis, Mo.) or glutathione derivatized microtiter plates, which are then combined with the cell lysates and the test modulating agent, and the mixture incubated under conditions conducive to complex formation, for example, at physiological conditions for salt and pH, though slightly more stringent conditions may be desired. Following incubation, the beads are washed to remove any unbound label, and the matrix immobilized and radiolabel determined directly (e.g., beads placed in scintillant), or in the supernatant after the complexes are subsequently dissociated. Alternatively, the complexes can be dissociated from the matrix, separated by SDS-PAGE, and the level of Ccdc80-binding protein found in the bead fraction quantitated from the gel using standard electrophoretic techniques.

Other techniques for immobilizing proteins on matrices are also available for use in the subject assay. For instance, Ccdc80 or a cognate binding protein thereof can be immobilized utilizing conjugation of biotin and streptavidin. Biotinylated Ccdc80 molecules can be prepared from biotin-NHS (N-hydroxy-succinimide) using techniques well known in the art (e.g., biotinylation kit, Pierce Biotechnology, Rockford, Ill.), and immobilized in the wells of streptavidin-coated 96 well plates (Pierce Biotechnology). Alternatively, antibodies reactive with Ccdc80 but which do not interfere with binding of upstream or downstream elements can be derivatized to the wells of the plate, and Ccdc80 trapped in the wells by antibody conjugation. As above, preparations of a Ccdc80-binding protein (Ccdc80-BP) and a test modulating agent are incubated in the Ccdc80-presenting wells of the plate, and the amount of complex trapped in the well can be quantitated. Exemplary methods for detecting such complexes, in addition to those described above for the GST-immobilized complexes, include immunodetection of complexes using antibodies reactive with the Ccdc80 binding element, or which are reactive with Ccdc80 protein and compete with the binding element, as well as enzyme-linked assays which rely on detecting an enzymatic activity associated with the binding element, either intrinsic or extrinsic activity. In the instance of the latter, the enzyme can be chemically conjugated or provided as a fusion protein with the Ccdc80 binding protein. To illustrate, the Ccdc80 binding protein can be chemically cross-linked or genetically fused with horseradish peroxidase, and the amount of protein trapped in the complex can be assessed with a chromogenic substrate of the enzyme, for example, 3,3′-diamino-benzadine terahydrochloride or 4-chloro-1-napthol. Likewise, a fusion protein comprising the protein and glutathione-S-transferase can be provided, and complex formation quantitated by detecting the GST activity using 1-chloro-2,4-dinitrobenzene (Habig W H et al., J. Biol. Chem. 249:7130-39 (1974)).

For processes that rely on immunodetection for quantitating one of the proteins trapped in the complex, antibodies against the protein, such as anti-Ccdc80 antibodies, can be used. Alternatively, the protein to be detected in the complex can be “epitope tagged” in the form of a fusion protein which includes, in addition to the Ccdc80 sequence, a second protein for which antibodies are readily available (e.g. from commercial sources). For instance, the GST fusion proteins described above can also be used for quantification of binding using antibodies against the GST moiety. Other useful epitope tags include myc-epitopes (see, e.g., Ellison M J and Hochstrasser M, J. Biol. Chem. 266:21150-57 (1991)) which includes a 10-residue sequence from c-myc, as well as the pFLAG® system (SigmaAldrich, St. Louis, Mo.) or the pEZZ-protein A system (GE Healthcare, Piscataway, N.J.).

It is also within the scope of this invention to determine the ability of a compound to modulate the interaction between Ccdc80 and its target molecules without the labeling of any of the interactants. For example, a microphysiometer can be used to detect the interaction of Ccdc80 with its target molecules without the labeling of Ccdc80 or the target molecules (see, e.g., McConnell H M et al., Science 257:1906-12 (1992)). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

In addition to cell-free assays, the readily available source of Ccdc80 proteins provided by the present invention also facilitates the generation of cell-based assays for identifying small molecule agonists/antagonists and the like. For example, cells can be caused to express or overexpress a recombinant Ccdc80 protein in the presence and absence of a test modulating agent of interest, with the assay scoring for modulation in Ccdc80 responses by the target cell mediated by the test agent. For example, as with the cell-free assays, modulating agents which produce a statistically significant change in Ccdc80-dependent responses (either an increase or decrease) can be identified.

Recombinant expression vectors that can be used for expression of Ccdc80 are known in the art (see discussions above). In one embodiment, within the expression vector the Ccdc80-coding sequences are operably linked to regulatory sequences that allow for constitutive or inducible expression of Ccdc80 in the indicator cell(s). Use of a recombinant expression vector that allows for constitutive or inducible expression of Ccdc80 in a cell is preferred for identification of compounds that enhance or inhibit the activity of Ccdc80. In an alternate embodiment, within the expression vector, the Ccdc80 coding sequences are operably linked to regulatory sequences of the endogenous Ccdc80 gene (i.e., the promoter regulatory region derived from the endogenous gene). Use of a recombinant expression vector in which Ccdc80 expression is controlled by the endogenous regulatory sequences is preferred for identification of compounds that enhance or inhibit the transcriptional expression of Ccdc80. In one embodiment, an assay is a cell-based assay comprising contacting a cell expressing a Ccdc80 target molecule (e.g., a Ccdc80 intracellular interacting molecule) with a test compound and determining the ability of the test compound to modulate (e.g. stimulate or inhibit) the activity of the Ccdc80 target molecule. Determining the ability of the test compound to modulate the activity of a Ccdc80 target molecule can be accomplished, for example, by determining the ability of the Ccdc80 protein to bind to or interact with the Ccdc80 target molecule or its ligand.

In an illustrative embodiment, the expression or activity of Ccdc80 is modulated in cells and the effects of modulating agents of interest on the readout of interest (such as, e.g., osteoblast activity or bone remodeling) are measured and/or observed.

In another embodiment, determining the ability of a Ccdc80 modulator to bind to or interact with a target molecule can be accomplished by measuring a read out of the activity of Ccdc80 or of the target molecule. For example, the activity of Ccdc80 or a target molecule can be determined by detecting induction of a cellular second messenger of the target, detecting catalytic/enzymatic activity of the target an appropriate substrate, detecting the induction of a reporter gene (comprising a target-responsive regulatory element operably linked to a nucleic acid encoding a detectable marker, e.g., chloramphenicol acetyl transferase), or detecting a target-regulated cellular response, for example, osteoblast activity or bone remodeling.

This invention further pertains to novel Ccdc80 modulators identified by the above-described screening assays. Accordingly, it is within the scope of this invention to further use Ccdc80 modulators identified as described herein in an appropriate animal model. For example, Ccdc80 modulators identified as described herein (e.g., a Ccdc80 modulating agent or a Ccdc80-binding partner) can be used in an animal model to determine the efficacy, toxicity, or side effects of treatment with such Ccdc80 modulators. Alternatively, Ccdc80 modulators identified as described herein can be used in an animal model to determine the mechanism of action of such Ccdc80 modulators. Furthermore, this invention pertains to uses of novel Ccdc80 modulators identified by the above-described screening assays for treatments as described herein.

### VII. ADMINISTRATION OF CCDC80 MODULATORS

Ccdc80 modulators are administered to subjects in a biologically compatible form suitable for pharmaceutical administration in vivo to either enhance or suppress Ccdc80 activity. By “biologically compatible form suitable for administration in vivo” is meant a form of the Ccdc80 modulator to be administered in which any toxic effects are outweighed by the therapeutic effects of the modulator. The term subject is intended to include living organisms in which an immune response can be elicited, for example, mammals. Administration of Ccdc80 modulators as described herein can be in any pharmacological form including a therapeutically active amount of an agent alone or in combination with a pharmaceutically acceptable carrier.

Administration of a therapeutically active amount of the Ccdc80 modulators of the present invention is defined as an amount effective, at dosages and for periods of time necessary, to achieve the desired result. For example, a therapeutically active amount of a Ccdc80 modulator may vary according to factors such as the disease state, age, sex, and weight of the individual, and the ability of peptide to elicit a desired response in the individual. Dosage regima may be adjusted to provide the optimum therapeutic response. For example, several divided doses may be administered daily, or the dose may be proportionally reduced as indicated by the exigencies of the therapeutic situation.

The therapeutic or pharmaceutical compositions of the present invention can be administered by any suitable route known in the art including, for example, intravenous, subcutaneous, intramuscular, transdermal, intrathecal, or intracerebral or administration to cells in ex vivo treatment protocols.

Ccdc80 modulators can also be linked or conjugated with agents that provide desirable pharmaceutical or pharmacodynamic properties. For example, Ccdc80 modulators can be coupled to any substance known in the art to promote penetration or transport across the blood-brain barrier such as an antibody to the transferrin receptor, and administered by intravenous injection (see, e.g., Friden P M et al., Science 259:373-77 (1993)). Furthermore, Ccdc80 modulators can be stably linked to a polymer such as polyethylene glycol to obtain desirable properties of solubility, stability, half-life, and other pharmaceutically advantageous properties (see, e.g., Davis et al., Enzyme Eng. 4:169-73 (1978); Burnham N L, Am. J. Hosp. Pharm. 51:210-18 (1994)).

Furthermore, Ccdc80 modulators can be in a composition which aids in delivery into the cytosol of a cell. For example, a Ccdc80 modulator may be conjugated with a carrier moiety such as a liposome that is capable of delivering the peptide into the cytosol of a cell. Such methods are well known in the art (see, e.g., Amselem S et al., Chem. Phys. Lipids 64:219-37 (1993)). Alternatively, a Ccdc80 modulator can be modified to include specific transit peptides or fused to such transit peptides which are capable of delivering the Ccdc80 modulator into a cell. In addition, the modulator can be delivered directly into a cell by microinjection.

The Ccdc80 modulators are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. Ccdc80 modulators can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion by continuous or periodic infusion.

Dose administration can be repeated depending upon the pharmacokinetic parameters of the dosage formulation and the route of administration used.

It is also provided that certain formulations containing the Ccdc80 modulators are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents, or flavoring agents. The compositions may be formulated so as to provide rapid, sustained, or delayed release of the active ingredients after administration to the patient by employing procedures well known in the art. The formulations can also contain substances that diminish proteolytic degradation and/or substances which promote absorption such as, for example, surface active agents.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the Ccdc80 modulator and the particular therapeutic effect to be achieved and (b) the limitations inherent in the art of compounding such an active compound for the treatment of sensitivity in individuals. The specific dose can be readily calculated by one of ordinary skill in the art, e.g., according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

Toxicity and therapeutic efficacy of such Ccdc80 modulators can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Ccdc80 modulators which exhibit large therapeutic indices are preferred. While Ccdc80 modulators that exhibit toxic side effects may be used, care should be taken to design a delivery system that targets such modulators to the site of affected tissue in order to minimize potential damage to uninfected cells and, thereby, reduce side effects.

The data obtained from the cell culture assays and animal studies can be used in formulating a range of dosage for use in humans. The dosage of such Ccdc80 modulators lies preferably within a range of circulating concentrations that include the ED₅₀ with little or no toxicity. The dosage may vary within this range depending upon the dosage form employed and the route of administration utilized. For any Ccdc80 modulator used in the method of the invention, the therapeutically effective dose can be estimated initially from cell culture assays. A dose may be formulated in animal models to achieve a circulating plasma concentration range that includes the IC₅₀ (i.e., the concentration of the Ccdc80 modulator that achieves a half-maximal inhibition of symptoms) as determined in cell culture. Such information can be used to more accurately determine useful doses in humans. Levels in plasma may be measured, for example, by high performance liquid chromatography.

In one embodiment of this invention, a Ccdc80 polypeptide may be therapeutically administered by implanting into patients vectors or cells capable of producing a biologically-active form of Ccdc80 or a precursor of Ccdc80, that is, a molecule that can be readily converted to a biological-active form of Ccdc80 by the body. Similar methods of administering may be used in cosmetic treatment of subjects.

In one approach, cells that secrete Ccdc80 may be encapsulated into semipermeable membranes for implantation into a patient. The cells can be cells that normally express Ccdc80 or a precursor thereof or the cells can be transformed to express Ccdc80 or a biologically active fragment thereof or a precursor thereof. It is preferred that the cell be of human origin. However, the formulations and methods herein can be used for veterinary as well as human applications and the term “patient” or “subject” as used herein is intended to include human and veterinary patients.

Monitoring the influence of Ccdc80 modulators on the expression or activity of a Ccdc80 protein can be applied not only in basic drug screening, but also in clinical trials. For example, the effectiveness of a Ccdc80 modulator determined by a screening assay as described herein to increase Ccdc80 gene expression, protein levels, or upregulate Ccdc80 activity, can be monitored in clinical trials of subjects exhibiting decreased Ccdc80 gene expression, protein levels, or downregulated Ccdc80 activity. In such clinical trials, the expression or activity of a Ccdc80 gene, and preferably other genes that have been implicated in a disorder can be used as a “read out” or markers of the phenotype of a particular cell.

For example, and not by way of limitation, genes, including Ccdc80, that are modulated in cells by treatment with a Ccdc80 modulator (e.g., compound, drug, or small molecule) that modulates Ccdc80 activity (e.g., identified in a screening assay as described herein) can be identified. Thus, to study the effect of agents on a Ccdc80 associated disorder, for example, in a clinical trial, cells can be isolated and RNA prepared and analyzed for the levels of expression of Ccdc80 and other genes implicated in the Ccdc80-associated disorder, respectively. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of Ccdc80 or other genes. In this way, the gene expression pattern can serve as a marker, indicative of the physiological response of the cells to the agent. Accordingly, this response state may be determined before, and at various points during, treatment of the individual with the Ccdc80 modulator.

The present invention also provides a method for monitoring the effectiveness of treatment of a subject with a Ccdc80 modulator (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the Ccdc80 modulator; (ii) detecting the level of expression of a Ccdc80 protein, mRNA, or genomic DNA in the pre-administration sample; (iii) obtaining one or more post-administration samples from the subject; (iv) detecting the level of expression or activity of the Ccdc80 protein, mRNA, or genomic DNA in the post-administration samples; (v) comparing the level of expression or activity of the Ccdc80 protein, mRNA, or genomic DNA in the pre-administration sample with the Ccdc80 protein, mRNA, or genomic DNA in the post administration sample or samples; and (vi) altering the administration of the Ccdc80 modulator to the subject accordingly. For example, increased administration of the Ccdc80 modulator may be desirable to increase the expression or activity of Ccdc80 to higher levels than detected, that is, to increase the effectiveness of the agent. Alternatively, decreased administration of the agent may be desirable to decrease expression or activity of Ccdc80 to lower levels than detected, that is, to decrease the effectiveness of the Ccdc80 modulator. According to such an embodiment, Ccdc80 expression or activity may be used as an indicator of the effectiveness of a Ccdc80 modulator, even in the absence of an observable phenotypic response.

In a preferred embodiment, the ability of a Ccdc80 modulator to modulate osteoblast activity and/or bone remodeling in a subject that would benefit from modulation of the expression and/or activity of Ccdc80 can be measured by detecting an improvement in the condition of the patient after the administration of the Ccdc80 modulator. Such improvement can be readily measured by one of ordinary skill in the art using indicators appropriate for the specific condition of the patient. Monitoring the response of the patient by measuring changes in the condition of the patient is preferred in situations were the collection of biopsy materials would pose an increased risk and/or detriment to the patient.

Furthermore, in the treatment of disease conditions, compositions containing Ccdc80 can be administered exogenously and it would likely be desirable to achieve certain target levels of Ccdc80 polypeptide in sera, in any desired tissue compartment, or in the affected tissue. It would, therefore, be advantageous to be able to monitor the levels of Ccdc80 polypeptide in a patient or in a biological sample including a tissue biopsy sample obtained from a patient and, in some cases, also monitoring the levels of native Ccdc80. Accordingly, the present invention also provides methods for detecting the presence of Ccdc80 in a sample from a patient.

### EXAMPLES

The present invention is further defined in the following Examples. It should be understood that these Examples, while indicating preferred embodiments of the invention, are given by way of illustration only. From the above discussion and these Examples, one skilled in the art can ascertain the preferred features of this invention, and without departing from the spirit and scope thereof, can make various changes and modification of the invention to adapt it to various uses and conditions.

### Example 1

To demonstrate that Ccdc80 is a secreted protein, Applicants cloned the open reading frame of human Ccdc80 (sequence identical to GenBank® Accession No. NM_{—}199511) fused to a C-terminal flag tag into the mammalian expression vector pSMED2. The following primers were used:

Forward

(SEQ ID NO:1)

5′-ACGCTGTCGACCACCGCAACCCTCTGCATTCCATCTC-3′
Reverse

(SEQ ID NO:2)

5′-CGTCTAGATTCACTTATCGTCGTCATCCTTGTAATCGTAAGGGTATC
CATGGTGATAACTC-3′

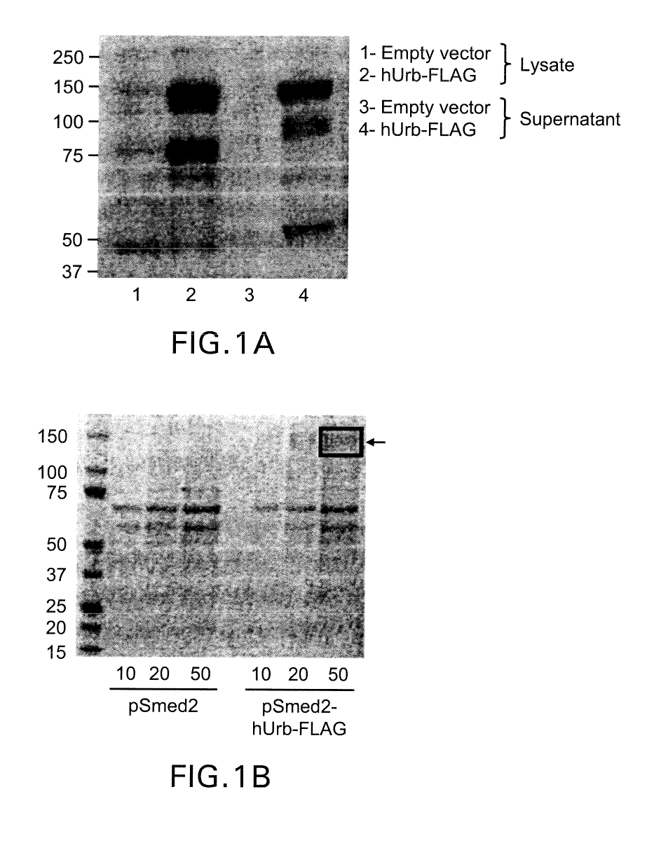

The Ccdc80-Flag containing expression vector as well as a control vector were transfected into HEK293T cells. 48 hours after transfection, the cells were switched into serum-free medium, and 24 hours later, cells and supernatant were collected. Cells were lysed in lysis buffer (20 mM Tris, pH 7.4; 140 mM NaCl, 1 mM CaCl₂; 1 mM MgCl₂; 10% glycerol; 1% NP-40; protease inhibitors), and the supernatant was spun at 2,500 rpm for 10 minutes to remove debris. Western blotting using anti-Flag antibody (FIG. 1A) shows that Flag-reactive material is present in both the cell lysate and the supernatant. The majority of the Flag-reactive material is present in an ˜150 kDa band, similar to what had been described before for mouse Ccdc80. Commassie staining of the supernatant (FIG. 1B) shows a prominent ˜150 kDa band (arrow) that is present only in the supernatant of Ccdc80-transfected cells. This band was identified as human Ccdc80 by mass spectroscopy. These data demonstrate that human Ccdc80 is indeed a secreted protein.

To examine the function of Ccdc80 in bone formation in subsequent experiments, Applicants used conditioned supernatant from Ccdc80- and control-transfected cells (FIG. 1B). Applicants also purified Ccdc80 from conditioned medium using Flag affinity beads. Briefly, Ccdc80 was immunoprecipitated using anti-FLAG M2 affinity beads (˜1 ml of beads/50 ml supernatant) overnight at 4° C. Immune complexes were washed 3 times with Tris-buffered saline (TBS), then FLAG-tagged Ccdc80 was eluted from the beads using 5 ml of 3×FLAG peptide solution (150 μg/ml of TBS) and kept at −20° C.

### Example 2

### Neonatal Calvaria Organ Culture Assay

Applicants used mouse neonatal calvaria organ culture assay to examine possible roles for Ccdc80 in bone formation/resorption. Neonatal mouse calvaria were prepared from 4-day-old pups as described previously (Traianedes K et al., Endocrinology 139:3178-84 (1998); Mundy G et al., Science 286:1946-49 (1999)). Briefly, calvaria were excised and cut in half along the sagittal suture. Each half calvaria was placed with the concave surface downward on a stainless steel grid (Small Parts, Inc., Miami, Fla.) in a 12-well tissue culture dish (Becton Dickinson, Oxnard, Calif.). Calvaria were pre-incubated overnight in serum-free BGJ medium with 0.1% BSA. Each well contained 1 ml of BGJ medium. After pre-incubation, calvaria were incubated for 7 days in BGJ medium containing 1% FBS without or with test samples. Calvaria in 12-well plates were incubated in a humidified atmosphere of 95% air and 5% CO₂. Fresh test samples were added at the time of medium change on day 4.

After organ culture, calvaria were fixed in 10% neutral phosphate-buffered formaldehyde at room temperature for 72 hours, then decalcified for 5-6 hours in 10% EDTA in PBS. Calvaria in each group were embedded in parallel in the same paraffin block, and 4 μm sections were stained with hematoxylin-eosin. Consistent bone areas (200 μm away from frontal sutures) were selected for histomorphometric analysis. In brief, a 200 μm square grid was placed on each calvaria, and the number of osteoblasts and total bone area were determined with the Osteomeasure System (Osteometrics Inc., Atlanta, Ga.). All cells on the bone surface were counted as osteoblasts.

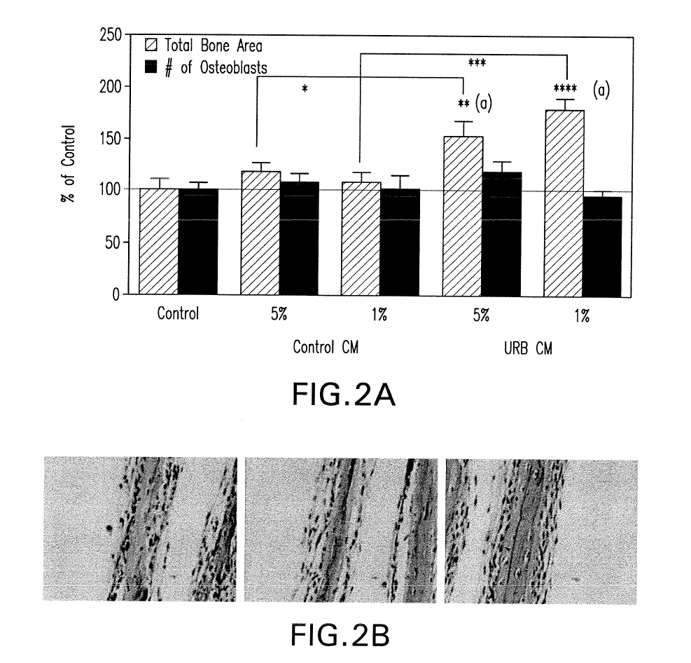

As shown in FIG. 2A, incubation with 1% or 5% Ccdc80 conditioned medium (CM) for 7 days resulted in a significant increase in total bone area by 79% (P<0.0001) and 53% (P<0.01), respectively (see FIG. 2A and FIG. 2B). Although the number of osteoblasts was not altered, osteoblasts became cuboidal in shape, suggesting that they were activated. Previous studies have shown that activation of osteoblasts is capable of supporting increased bone formation (Traianedes K et al., Endocrinology 139:3178-84 (1998); Mundy G et al., Science 286:1946-49 (1999)). Neither total bone area nor number or morphology of osteoblasts was significantly altered by incubation with control CM (FIG. 2A).

### Example 3

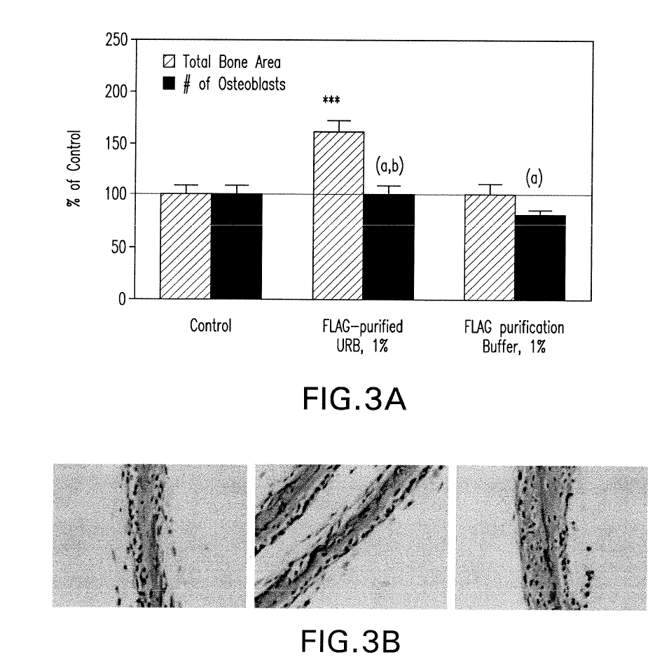

To confirm that the osteogenic effects of Ccdc80 CM were due to Ccdc80 protein, Applicants examined actions/activities of FLAG-purified Ccdc80 protein (FIG. 3A). Incubation of calvaria, prepared as described in Example 2, with 1% FLAG-purified Ccdc80 protein for 7 days resulted in a 62% increase in total bone area (P<0.0001). Although the number of osteoblasts was not significantly altered, osteoblasts were activated (FIG. 3A and FIG. 3B). Histologically, enhanced bone resorption also was evident (FIG. 3B). Together, it is most likely that Ccdc80 protein enhanced bone remodeling (both bone formation and bone resorption) in favor of bone formation.

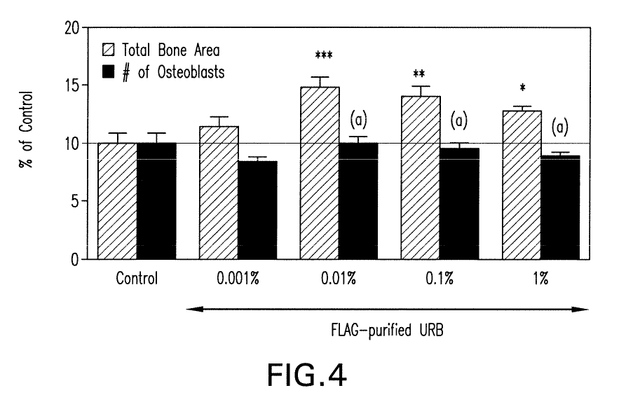

Osteogenic activities of FLAG-purified Ccdc80 protein were clearly observed when the purified Ccdc80 protein sample was diluted as low as 0.01% (see FIG. 4). For this experiment, calvaria were prepared from 4-day old neonatal pups. Calvaria then were incubated for 7 days without (Control), or with serial dilutions of FLAG-purified Ccdc80 protein as shown in FIG. 4. Medium was changed on day 4. After organ culture, calvaria were fixed in 10% neutral phosphate buffered formaldehyde, embedded in paraffin block, sectioned at 4 μm, and stained with hematoxylin and Eosin for histomorphoemetric analysis.

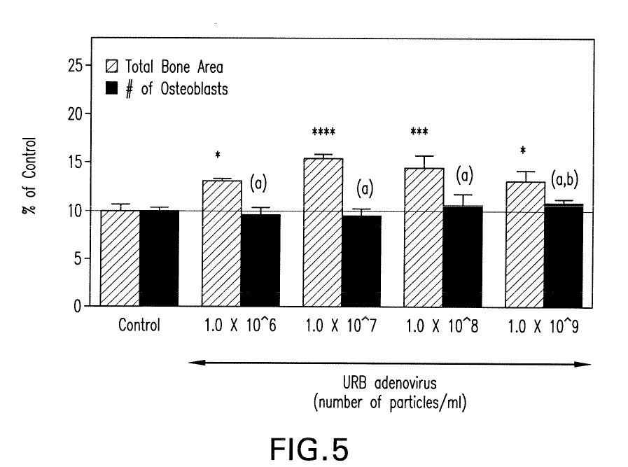

In FIG. 5, the effects of Ccdc80 gene transfer on total bone area and number of osteoblasts is shown. For this experiment, calvaria were prepared from 4-day old neonatal pups. Calvaria then were infected without (Control) or with various particle numbers of Ccdc80-adenovirus as shown in FIG. 5. Fresh treatments were added when medium was changed on day 4. After organ culture, calvaria were fixed in 10% neutral phosphate buffered formaldehyde, embedded in paraffin block, sectioned at 4 mm, and stained with hematoxylin and Eosin for histomorphoemetric analysis.

All of these findings are consistent with the idea that Ccdc80 protein is osteogenic.

### Example 4

As described above, BMP-2 is a well-known osteogenic factor and is used clinically as a bone-inductive agent. It has also been shown that osteogenic effects of strains, cholesterol-lowering drugs, involve up-regulation of endogenous BMP-2 (Chowdhury J R et al., Science 254:1802-05 (1991)). Thus, Applicants next examined if stimulatory effects of Ccdc80 on bone formation might involve actions/activities of BMP-2. To do this, C2C12 cells were transiently transfected with plasmids containing either a BMP-response element fused to a luciferase reporter gene (to monitor BMP-dependent transcriptional activity) or a response element containing binding sites for the TCF/LEF transcription factors fused to a luciferase reporter gene (to monitor β-catenin-dependent transcriptional activity). After transfection, cells were infected with an adenovirus encoding either GFP or human Ccdc80 for 16 hrs, after which they were allowed to differentiate in low-mitogenic condition (reduction of serum from 10% to 1% FBS) in the absence or presence of BMP-2 (50 ng/ml) for 48 hrs. BMP- and β-catenin-mediated transcription were evaluated by measuring luciferase activity, and results were expressed relative to β-galactosidase (control).

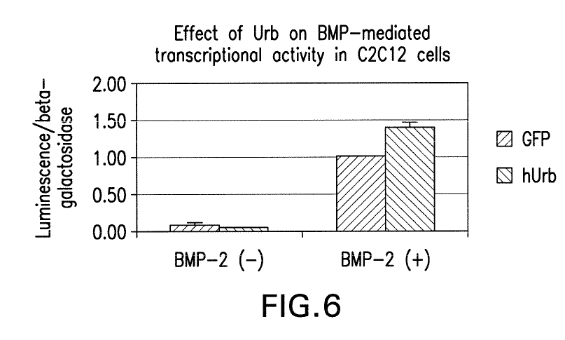

BMP-2 is a potent stimulator of bone remodeling, which is known to direct differentiation of C2C12 myoblasts into osteoblasts. As expected, addition of BMP-2 dramatically enhanced the transcriptional activity of the BMP-response element in C2C12 cells (FIG. 6). Expression of Ccdc80 per se was insufficient to activate the BMP-response element (FIG. 6). However, in the presence of BMP-2, Ccdc80 overexpression significantly enhanced (40%, p<0.05) the transcriptional signal above the level seen with BMP-2 alone (FIG. 6), suggesting that Ccdc80 may act by potentiating BMP-2 signaling. Applicants do not know at this time if these findings may constitute the mechanism by which Ccdc80 stimulated bone formation in ex vivo calvaria assays. Yet, cell-based assays described here should be particularly useful for studies to clone the putative receptor(s) for Ccdc80.

### Example 5

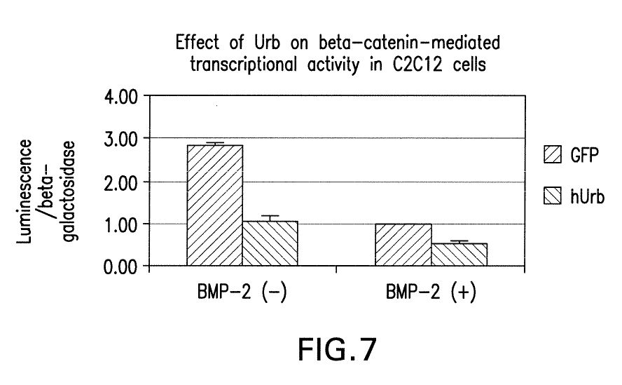

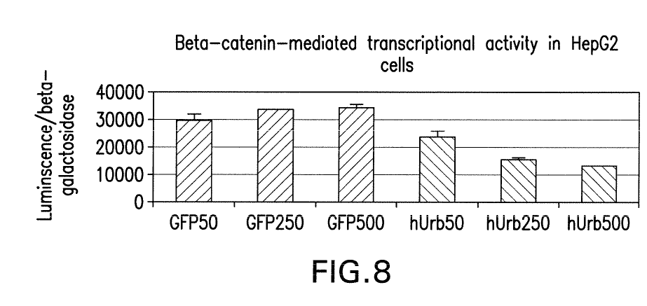

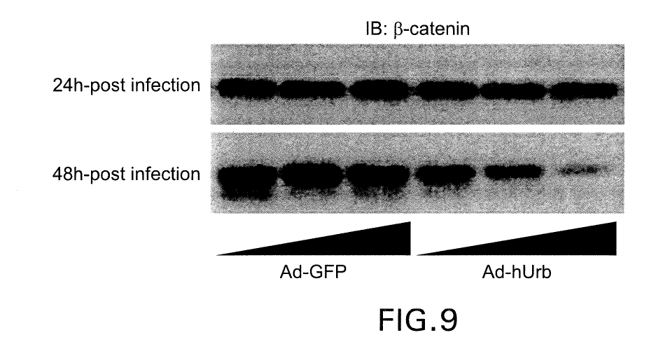

Applicants then determined the effect of Ccdc80 on β-catenin-mediated transcription, a known component of bone remodeling in a large number of experimental paradigms. As has been previously described, the osteogenic factor BMP-2 blunts β-catenin-mediated transcriptional activity (FIG. 7). Ccdc80 significantly decreased the reporter activity linked to the activation of the canonical β-catenin signaling pathway in both the absence and the presence of BMP-2 (FIG. 7). The repressing effect of Ccdc80 on β-catenin-mediated transcriptional activity in C2C12 cells was also confirmed in HepG2 hepatoma, a cell line that express a constitutively active form of β-catenin. Indeed, adenoviral expression of Ccdc80 dose-dependently decreased the β-catenin-dependent reporter activity when compared to cells expressing the control protein GFP (FIG. 8). In agreement with the latter observation, Applicants found that Ccdc80 decreased the protein expression of β-catenin in a time- and dose-dependent manner (FIG. 9).

### Example 6

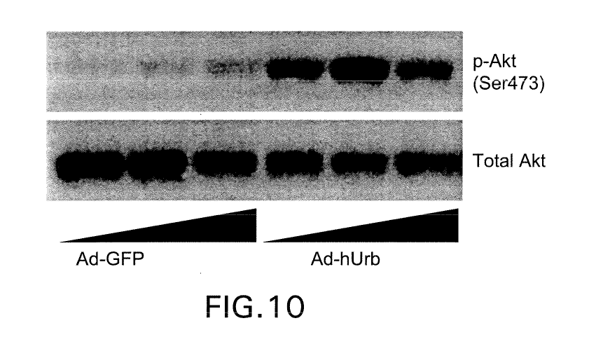

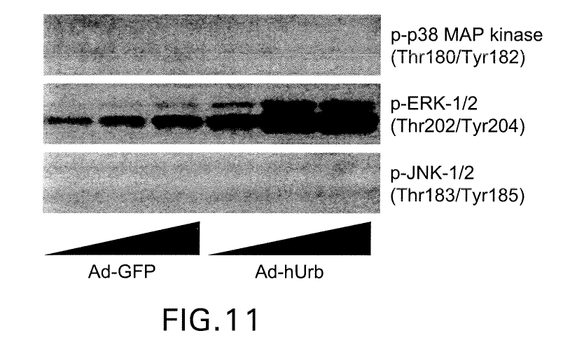

To further gain insights into the molecular mechanism(s) leading to Ccdc80-induced bone formation, Applicants analyzed the effect of Ccdc80 expression on two major signaling pathways that are linked to osteogenesis and bone remodeling, namely the Akt and MAPK pathways. Adenoviral overexpression of Ccdc80 in HepG2 cells leads to a dramatic increase in the phosphorylation of Akt (a measure of its activity) despite unchanged (or slightly lower) expression of total Akt (FIG. 10). Ccdc80 was also found to increase the phosphorylation of ERK-1/2, but not that of two other members of the MAPK family, p38 MAPK and c-Jun-N-terminal kinase (JNK) (FIG. 11).

What is claimed is:

1. A method for the treatment of a mammal suffering from a bone disorder comprising administering to the mammal in need thereof a therapeutically effective amount of an agent that modulates the expression or activity of the Ccdc80 gene or Ccdc80 protein.

2. The method of claim 1, wherein the bone disorder is osteoporosis, rickets, osteomalacia, chronic renal failure, hyperparathyroidism, osteopenia, Paget's disease, or a bone lesion.

3. The method of claim 1, wherein the mammal is a human.

4. The method of claim 1, wherein the agent is a compound, a protein, a polypeptide, an antibody, an aptamer, or a polynucleotide.

5. The method of claim 4, wherein the agent is a human, mouse, rat, or chicken Ccdc80 polynucleotide.

6. The method of claim 4, wherein the agent is a human, mouse, rat, or chicken Ccdc80 polypeptide.

7. The method of claim 4, wherein the agent is a Ccdc80 peptidomimetic or Ccdc80 agonist.

8. The method of claim 4, wherein the agent is a compound.

9. The method of claim 4, wherein the agent directly modulates the expression or activity of the Ccdc80 gene or Ccdc80 protein.

10. The method of claim 4, wherein the compound is rosiglitazone.

11. A method of identifying a Ccdc80 receptor comprising:
a) providing Ccdc80 polypeptide to a cell suspected of containing a Ccdc80 receptor;
b) identifying specific binding of the Ccdc80 polypeptide to the cell; and
c) isolating the source of the specific binding.

12. The method of claim 11, wherein the cell is an osteoblast, osteoclast, or hepatocyte.

13. The method of claim 11, wherein step a) is performed by expression of the Ccdc80 polypeptide in the cell.

14. The method of claim 13, wherein Ccdc80 polypeptide expression is from an expression vector comprising a Ccdc80 polynucleotide.

15. The method of claim 13, wherein the Ccdc80 polypeptide is secreted by the cell.

16. The method of claim 11, wherein step a) is performed by expression and secretion of the Ccdc80 polypeptide by a different cell.

17. The method of claim 11, wherein step a) is performed by contacting the Ccdc80 polypeptide with the cell.

18. The method of claim 11 performed in vivo.

19. The method of claim 11 performed in vitro.

20. A method of activating osteoblasts and/or enhancing bone remodeling comprising contacting an osteoblast with an effective amount of an agent that modulates the expression or activity of the Ccdc80 gene or Ccdc80 protein.

21. A method of screening for Ccdc80 mimics comprising:
a) providing a candidate mimic and a Ccdc80 polypeptide; and
b) determining whether the candidate mimic competes with Ccdc80 polypeptide in an assay designed to assess Ccdc80 polypeptide activity;
wherein the Ccdc80 polypeptide activity is activation of osteoblasts and/or enhancement of bone remodeling.

22. The method of claim 21, wherein the assay is performed in a calvaria organ culture.

23. The method of claim 21, wherein the assay is a measurement of total bone area.

24. The method of claim 21, wherein the assay is a measurement of osteoblast morphology.

25. A method of screening for modulators that affect Ccdc80 activity comprising:
a) providing a candidate modulator and a Ccdc80 polypeptide; and
b) determining whether the candidate modulator interferes with or enhances Ccdc80 activity; wherein the Ccdc80 activity is activation of osteoblasts and/or enhancement of bone remodeling.


SEQUENCE LISTING (2 sequences)

SEQ ID NO: 1 - DNA, 37 bp, Artificial
  Forward CCDC80 PCR Primer
acgctgtcgaccaccgcaaccctctgcattccatctc

SEQ ID NO: 2 - DNA, 61 bp, Artificial
  Reverse CCDC80 PCR Primer
cgtctagattcacttatcgtcgtcatccttgtaatcgtaagggtatccatggtgataactc